(19) United States
(12) Patent Application Publication
(10) Pub. No.: US 2007/0003954 A1
(43) Pub. Date: Jan. 4, 2007

(54) Protein and antibody profiling using small molecule microarrays

(75) Inventor: Thomas Kodadek, Dallas, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/433,069

(22) Filed: May 12, 2006

Related U.S. Application Data
(60) Provisional application No. 60/680,200, filed on May 12, 2005.

(51) Int. Cl. C12Q 1/68; G01N 33/53

(52) U.S. Cl. 435/6; 435/7.1

(57) ABSTRACT

Aspects of the present invention describe methodology by which arrays of synthetic molecules can be created and employed for various types of proteomics profiling experiments. The most important of these from a clinical standpoint are the visualization of antibody and T cell binding patterns, which could be employed as a tool for monitoring the state of the immune system of a patient. This may be a generally useful tool for the diagnosis of many types of disease states. Similar techniques are employed to detect the post-translational modification of specific proteins, a tool for the visualization of induction of signal transduction pathways in cells and tissues treated with drugs. Finally, aspects of the invention teache a method for the creation of simpler arrays with less than 100 features that are, nonetheless, effective for protein profiling experiments.

This application claims priority to U.S. Provisional Patent application Ser. No. 60/680,200, filed on May 12, 2005, entitled “Protein and antibody profiling using small molecule microarrays,” which is incorporated herein by reference in its entirety.

The United States Government own rights in the present invention pursuant to grant NO1-HV-28185 from the National Institute of Health (NIH) entitled “UT-Southwestem Center for Proteomics Research.”

### BACKGROUND OF THE INVENTION

I. Field of the Invention

The present invention relates generally to biochemistry, proteomics, and diagnostics. In particular, the invention relates to compositions and methods for profiling or fingerprinting proteins in a target sample, such as antibodies.

II. Background

The continuing advance of medical diagnostic technology is strongly dependent on the discovery of new biomarkers, defined broadly as proteins whose presence, absence, and/or chemical modification state serve as indicators (directly or indirectly) of a particular disease state or condition, how a patient reacts to a drug, etc. Unfortunately, the discovery of new biomarkers has proven challenging. There is considerable agreement in the field that one way to accelerate the development of new diagnostic tools is to expand beyond traditional “single biomarker” approaches to the consideration of several markers simultaneously. The idea is that even if one marker is not completely definitive, the combination of the levels and/or modification states of several markers would provide an unambiguous diagnosis. The question is how to accomplish this goal in a scientifically and economically reasonable way.

Most of the work done in this area has been directed toward the development of DNA microarrays as diagnostic tools. In this approach, RNA is isolated from an appropriate source, reverse transcribed into cDNA, labeled, and hybridized to a DNA microarray of the type sold by Affymetrix or other companies. The concept is that the pattern of gene expression in the sample will provide a “fingerprint,” “profile,” or “signature” of the state of the cell or organism that is being analyzed. However, the collection and labeling of RNA samples requires a high degree of technical skill and to achieve reproducibility.

Another approach that has been popularized recently is to employ mass spectrometry as a fingerprinting tool. For example, a sample is fractionated on a chip containing several different chromatography surfaces, allowing some number of proteins to be absorbed on each surface. These surfaces are then probed by MALDI (or SELDI) mass spectrometry. In the simplest version of this experiment, the peaks are treated simply as unidentified signals and the pattern of these signals is employed diagnostically. Although this approach generated considerable early excitement in the diagnosis of cancer, it has also suffered from significant reproducibility problems and requires expensive instrumentation.

A different methodology has been applied to the analysis of autoantibodies present in patients with autoimmune disorders or cancers. In this array-based approach, particular selected proteins or peptide antigens are immobilized on a suitable surface, such as a chemically-modified glass slide, and the serum of a patient is hybridized to the array. Binding of autoantibodies to these arrays is then measured through susequent application to the array of a labeled secondary antibody (e.g., anti Ig-G). A variant of this approach that is more suitable to diseases where the appropriate capture antigens are unknown or impractical to prepare is to begin with material from a paitent sample(s) and use this material or derivatives derived from it as the features on an antibody capture array. For example, one approach is to prepare extracts from tumor biopsies and fractionate them chromatographically. Each protein fraction is then spotted onto a microarray with the expectation that some will contain cancer-specific antigens. More recently, RNA from tumor samples has been collected and used to make a cDNA library that was then used, in turn, to construct a phage display library. The phage were then treated so as to enrich those that displayed proteins that bound antibodies in the serum of prostate cancer patients, but not healthy control patients. The viruses were then prepared in quantity and spotted down on an array to provide a diagnostic tool for the detection of antibodies enriched in the serum of prostate cancer patients. These approaches have provided interesting results, but no one has yet demonstrated that this constitutes a practical diagnostic tool for many different diseases, since the appropriate antigens that would be necessary to construct such an array are often unknown or difficult to produce. The common feature of all such approaches is that they endeavor to employ and/or discover native antigens that can act as “capture agents” for disease-sepcific antibodies when immobilized on an array surface or the equivalent.

Further limitations and disadvantages of conventional and traditional approaches will become apparent to one of skill in the art, through comparison of such systems with aspects of the present invention as set forth in the present application.

### SUMMARY OF THE INVENTION

The present invention provides methods of using synthetic molecules, i.e., ligands, that bind ligand binding moieties, such as proteins, nucleic acids, carbohydrates, or non-adherent cells present in complex biological mixtures, as biomarkers for a particular physiological state(s). The synthetic molecules may have not been previously selected to bind ligand binding moieties, which includes biomarkers present in a sample. In some cases the identities of ligand binding moieties known prior to the process. The invention includes methods comprising: (a) constructing an array of synthetic molecules having a plurality of structures; (b) contacting said array with a complex biological mixture obtained from animals or cells that exhibit a physiological state of interest, resulting in the capture of certain biological molecules or cells by certain molecules immobilized on the array; (c) assessing binding of certain captured molecules or cells to this array through the use of a labeled reagent that binds specifically to a given class of captured molecules or proteins; and (d) comparison of this binding pattern with the binding pattern of an appropriate control sample that does not represent the physiological state of interest. Aspects of the invention include constructing the array from synthetic molecules not previously selected to bind any particular molecule or cell in the sample of interest. In certain embodiments, the array of synthetic molecules is an array of peptoids (peptoid-like oligomers) derived from a combinatorial library. The complex biological mixture can be a serum sample obtained from an animal or patient with or suspected of having a disease. Binding of serum antibodies to the array is typically quantified by subsequent incubation with a fluorescently labeled secondary antibody. Peptoids that capture antibodies enriched in the diseased state are identified by comparison of the pattern of antibody binding of the two samples to the arrays.

Further embodiments of the invention include methods of detecting a plurality of distinct ligand binding moieties in a sample comprising (a) providing an array of ligands having a plurality of random structures; (b) contacting said array with a sample comprising ligand binding moiety; and (c) assessing binding of ligand binding moiety to said array, wherein binding of ligand binding moiety to said array detects ligand binding moieties in said sample. In certain aspects of the invention one or more ligand binding moiety is present in a body fluid or on a cell surface. In particular embodiments a ligand binding moiety is an antibody. Aspects of the invention include assessing binding of the ligand binding moiety to the array features by contacting the array with labeled or otherwise detectable anti-Ig, such as IgM, IgG, etc. In a further aspect, the ligand binding moieties can be a family of enzymes. The binding of this family of enzymes may be assessed using fluorescently labeled or otherwise labeled mechanism-based inhibitors or other covalent inhibitors. In a further aspect, the ligand binding moieties or biomarkers can be a class of non-adherent cells, such as T cells, and the binding pattern of the cells to the array could be detected by subsequent exposure of the array to a labeled antibody that recognizes a conserved molecule on the surface of cells in this family. In still further aspects, a ligand binding moiety can be a nucleotide-binding protein, a glycosylated protein, protein that share a post-translational modification, a peptide hormone or ligand, whose binding may be assessed by fluorescenty detectable or otherwise labeled nucleotides or nucleotide analogues, fluorescently or otherwise-labeled sugar-binding molecules, or fluorescently or otherwise-labeled antibodies.

The synthetic molecules displayed on the array (ligands) can include peptides, peptoids, oligonucleotides, oligosaccharides or small molecules not previously selected as ligands for specific target molecules. They also may comprise a common chemicl feture that prediagnosis binding to a particular class of ligand binding moieties.

In certain embodiments of the invention, the pattern of binding of the biomarkers or ligand binding moiety is predictive of a disease state in a subject from which said sample was obtained. Such a disease state can include, but is not limited to, cancer, autoimmune disease, inflammatory disease, infectious disease, neurodegenerative disease, and/or cardiovascular disease. In certain aspects, the ligands differentiate between different forms of a disease state, such as a mild or aggressive, or a chronic or progressive disease state. In a particular aspect the methods are capable of differentiating between a disease state that is or is not responsive to a treatment or therapy. In still further embodiments the molecules on the array (i.e., the ligands) capture potential biomarkers induced in breast cancer, lung cancer, prostate cancer, cervical cancer, head and neck cancer, testicular cancer, ovarian cancer, skin cancer, brain cancer, pancreatic cancer, liver cancer, stomach cancer, colon cancer, rectal cancer, esophageal cancer, lymphoma, or leukemia, such as antibodies that recognize epitopes unique to these disease states. In further aspects, the molecules on the array bind ligand binding moieties induced in lupus, myestenia gravis, multiple sclerosis, narcolepsy, rheumatoid arthritis, nephritis, Chagas disease, scleroderma, or Sjogren's disease. In still further apects, the molecules on the array bind ligand binding moieties induced as a result of infection with viruses, bacteria or fungi. In yet a further aspect, the molecules on the array bind ligand binding moieties induced by neurodegenerative diseases, including Alzheimer's disease, dementia, or Creutzfeld-Jacob disease.

Embodiments of the invention include methods where the synthetic molecules immobilized on the array will not have been previously selected for binding to a potential ligand binding moiety (i.e., the ligands will be structurally “random,” nonselected, or unbiased ligands), but may or may not contain structural elements that are anticipated to bias them towards binding to a given class of potential biomarkers. That is, the random ligands can comprise a purely random feature and/or a non-random feature. For example, in certain embodiments, all of the synthetic molecules on the array would contain, in addition to other chemical moieties, a purine analogue, which is anticipated to bias the compounds towards capturing ATP-binding proteins such as protein kinases.

An array of synthetic molecules can include 1000, 2,000, 4,000, 6,000, 7,000, 8,000 and 10,000, 12,500, 15,000, 25,000, 50,000, 100,000 or more distinct chemical species or random ligands, including the various values and ranges there between. An array can be, but is not limited to, a glass slide, a microscope slide, a plate, a chip, or a population of beads. The method may include cross-linking the ligand binding moiety to the array. One or more molecules on the array can be associated with binding to a ligand binding moiety, i.e., smart or focued array. The array, which is otherwise comprised of molecules not previously selected for particular binding properties, may also contain several known ligands for particular molecules in the complex biological sample. These binding events would serve as controls to evaluate the quality of the array.

Aspects of the invention include assessment of one or more samples including, but not limited to, urine, serum, whole blood, cerebrospinal fluid, sputum, stool, saliva, and semen. A sample can be obtained from a variety of organisms, including, but not limited to, a domestic animal, a cow, a horse, a bird, a chicken, or a human.

Embodiments of the invention can also include methods for detecting the binding of one or more isoforms of a ligand binding moiety in a sample involving (a) providing an array having a plurality of immobilized synthetic molecules not previously selected to bind a ligand binding moiety or moieties; (b) contacting said array with a sample containing one or more isoforms of the ligand binding moiety; and (c) assessing binding of one or more isoforms to the array, wherein binding of one or more isoforms detects one or more isoforms in the sample. For example, in one aspect, the complex biological mixture is a cell extract prepared from cells that have been stimulated with a chemical. The binding pattern of a particular signal transduction protein to an array is visualized by incubation with a labeled antibody that recognizes multiple forms of that signal transduction protein. This binding pattern is then compared with that of the signal transduction protein present in unstimulated cells. If the two binding patterns differ substantially, it can be concluded that stimulation resulted in activation of that signaling pathway and post-translational modifications of the signal transduction factor, which altered its binding pattern to the array. The one or more isoforms can be phosphorylation isoforms, glycosylation isoforms, myristoylation isoforms, length isoforms, amino acid substitution isoforms, ubiquitylation isoforms, SUMOylation isoforms, NEDDylation isoforms, splice variants, methylation isoforms, acetylation isoforms, citrullation isoforms, nitrosylation isoforms, and/or formylation isoforms. Binding of these isoforms can be assessed by photometric or non-photometric means. The isoforms from multiple array assessments may be comapred with each other.

In a further embodiment, random ligands are peptides, peptoids, oligonucleotides, oligosaccharides, amino acid derivatives, or small molecules. The random ligands may be preselected based on known reactivity to said isoforms. Typically, the pattern of binding of one or more isoforms is predictive of a disease state in a subject from which a sample was obtained. The pattern of binding of the one or more isoforms can be predictive of activation or inhibition of a cellular pathway. In other aspects, the random ligands are not preselected based on known reactivity to said one or more isoforms.

In still further embodiments, a sample can be expose to a stimulant or stimulated prior to detecting binding of a ligand binding moiety. The sample or the source of the sample can be stimulated with a drug or is stimulated by an environmental condition, such as light, heat, cold, sleep deprivation, elevated noise, sound deprivation, light deprivation, or chemical exposure. The sample can comprise cells stimulated in vitro. In one aspect, the sample is obtained from a subject suffering from, suspected of having, or at risk of having or developing a disease or disease state.

Aspects of the present invention can be found in a method and system for creating and employing arrays of synthetic molecules for various types of proteomics profiling experiments, as shown in and/or described in connection with at least one of the figures, and as set forth more completely in the claims.

Aspects of the invention provide a platform for the determination of “immune signatures.” This refers to the pattern of binding of antibodies or T cells to an array of synthetic compounds.

Further aspects of the invention provide a method for determining if signal transduction pathways have been activated, for example by treatment with a drug. This is done by hybridizing a cell extract to an array of synthetic compounds, then visualizing the binding pattern of a particular protein kinase specifically by hybridization with a labeled antibody. A phosphorylated (activated) kinase provides a different pattern than does an unactivated kinase.

Aspects of the invention provide a method for the discovery of synthetic molecules that act as particularly “information-rich” features in a microarray. These molecules, which are “promiscuous ligands” that bind to many proteins, can be used to create much simpler synthetic molecule arrays with far fewer features that are nonetheless quite effective for profiling experiments. Less than 100 to 75 promiscuous ligands may be used in profiling a sample. Studies have shown that 62 of 75 promiscuous ligand can bind a particular protein and produce a unique profile.

Aspects of the invention illustrate arrays comprised of several thousand peptides, peptoids or other synthetic molecules are capable of supporting such “protein fingerprinting” experiments.

Again, a basic concept underlying the invention is illustrated in FIG. 8. If one creates an array of several thousand synthetic molecules, then any protein hybridized to this array should bind to each feature of the array with a particular affinity and specificity. On most features, binding will not be detectable above background whereas a few features will bind the protein tightly. There will also be a certain number of features that will bind the protein at levels detectable above background, but less avidly than the few high affinity spots. The predicted outcome of this experiment is a unique pattern of binding of a given protein to the array. This is a “three-dimensional pattern” in that one quantifies binding of the proteins to the two-dimensional array, thereby providing a third dimension of information.

Even if thousands of proteins bind to the array of molecules, as would be the case if one hybridized to the array a complex sample such as serum or a cell extract, generally only the binding events of the protein of interest could be visualized selectively if one also hybridizes to the array a “sandwich reagent” such a labeled antibody that is highly specific for that protein (FIG. 8). Of course, the labeled antibody itself will evince a specific pattern of binding to the array. This pattern is measured in a separate control experiment and subtracted from the experimental data set. The inventors have recently demonstrated this approach.

It is claim that this technique produces different binding patterns on the array for different forms of the same protein, since these are chemically distinct species. If different forms of a protein result in different patterns, then one could distinguish these using only a single, general antibody that need not distinguish between different forms of the protein.

Another aspect of this technique is to measure many different proteins of the same class simultaneously. A good example of this approach is antibody profiling. All antibodies are quite similar, but have divergent antigen binding sites. Thus, any particular antibody is expected to provide a pattern that is unique, though there would be some overlap between the patterns. The binding of any antibody to the array is visualized by using a labeled anti-IgG. If a given antibody provides a specific pattern, then a group of antibodies evinces a particular “superpattern”. This can be an important diagnostic tool, since it is reasonable to assume that the immune system of an individual will react to a variety of maladies (cancer, infectious disease, atherosclerosis, sleep disorders, etc.) in a unique way. Given a sufficient number of retrospective studies to couple particular antibody signatures with specific disease states, this same diagnostic protocol could be employed to detect (clinically and pre-clinically) a large variety of medical conditions.

There are at least two major applications with significant implications to clinical medicine. First, with regard to antibody profiling, any disease which results in a significant change in the complement of antibodies could be diagnosed in this fashion. This would obviously include autoimmune diseases, cancer and several others.

The second application is a facile tool for mapping the activation of signal transduction cascades. This is extremely valuable to pharmaceutical companies in assessing the response of patients to drugs in clinical trials. In this manifestation, cells from the patient are lysed and hybridized to the chip, then probed with labeled antibodies raised against a protein kinase in the pathway of interest. The idea is that the profile of the kinase is different whether or not it had been activated by phosphorylation. This obviates the requirement for a difficult to obtain phospho-specific antibody. A series of these experiments is done using antibodies raised against kinases involved in different signaling pathways.

Methods may include the step of profiling the complement of any family of antibodies (IgG, IgM, etc.) in a biological sample (serum, blood, CSF, etc) by hybridization of that sample to an array of synthetic molecules followed by addition of a labeled antibody that recognizes any member of that antibody class (anti-IgG, anti-IgM, etc.).

Further methods include a step for profiling the complement of T cells in a biological sample by hybridization of that sample to an array of synthetic molecules followed by addition of a labeled antibody that recognizes a suitable cell surface marker present on the T cells, such as CD42 or others.

Still further methods include a step for detecting the activation of specific signal transduction pathways in cells by monitoring the binding pattern of a protein kinase involved in said pathway through hybridization of an extract to an array of synthetic molecules. Wherein, this binding pattern may be visualized specifically through the secondary hybridization of an antibody specific for said protein kinase followed by a labeled secondary antibody.

Method may also include the step of identify “promiscuous protein ligands” that are of utility in the construction of simplified, yet effective, protein fingerprinting arrays.

Other embodiments of the invention are discussed throughout this application. Any embodiment discussed with respect to one aspect of the invention applies to other aspects of the invention. The embodiments in the Examples section are understood to be embodiments of the invention that are applicable to all aspects of the invention. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the specification or claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates schematically how an exemplary synthetic molecule microarray is produced. A combinatorial library of compounds (in this case peptoids) is synthesized using the split and pool methodology, The beads are separated into the wells of microtiter plates where the compound is released from the bead into solution. A robotic spotter is then used to print the molecules onto a chemically-modified glass slide covalently. For peptoid microarrays, the identity of any particular molecule on the array can be determined by Edman degradation or mass spectrometry by going back to the appropriate well on the mother plate.

FIGS. 2A-2C illustrate protein profiling using a peptoid microarray. Images obtained by incubating fluorescently labeled Glutathione-S-Transferase (GST) or Ubiquitin (Ub) to a peptoid microarray containing 7680 different compounds (FIG. 2A). These images were obtained by scanning the arrays with a standard commercial array scanner used for DNA microarray analysis after hybridization and washing. The insets provide a magnified view to illustrate the signal to noise ratio obtained in the hybridization of 500 nM labeled protein to the array. On the right side of the figure are shown scatter plots that compare two independent runs of the same sample (GST1 vs. GST-2) (FIG. 2B) and two different proteins (GST vs. Ub) (FIG. 2C). Clusters of off-diagnonal points represent peptoid features that bind GST much better than Ub or vice versa. These plots demonstrate the reproducibility of the protein profiling method and the ability of the array to discriminate between different proteins. The “off-diagonal” features clearly cluster into two groups, one of which registers a much higher signal intensity in the antibody-utilizing experiment while the other provides a much higher signal in the experiment utilizing chemically labeled GST.

FIG. 3 shows a comparison of the binding patterns of two different monoclonal antibodies (anti-FLAG and anti-Myc) and demonstrate that these can be differentiated easily by the array.

FIG. 4 shows a cartoon illustrating the concept of antibody profiling as a diagnostic tool. The Y shaped molecules represent antibodies in the blood. Each of the antibodies will have a unique pattern of binding to the array. The entire “superpattern” produced by exposing the array to a serum sample and then visualizing it by subsequent incubation with fluorescently labeled anti-IgG secondary antibody, will be comprised of the sum of all of the individual antibody binding patterns weighted by their relative abundance in the serum. If, in a patient with a particular disease, the immune system responds by greatly amplifying a particular antibody, then the features to which that antibody binds will become corresponding brighter. By comparison of the pattern of antibody binding pattern from healthy controls with those from patients with a given disease, we can identify peptoids that capture disease-amplified antibodies. This invention eliminates the need to know anything about a native antigen so that one can use it as a capture agent to make an array, as well as the need to know exactly what protein or antibody one wishes to bind. The appropriate antibody-peptoid pairs are identified by comparison of the samples.

FIG. 5 shows a cartoon of the experiment done to test the utility of antibody profiling using an unselected peptoid array to detect EAE, the mouse model for multiple sclerosis. The disease was initiated by injection of mice with large amounts of a peptide antigen from mouse myelin basic protein, a nerve sheath component. Control mice were injected with saline. Serum was taken from the mice at various times after injection and the antibody pattern was analyzed on the peptoid microarrays.

FIG. 6 shows the results of an analysis of an EAE mouse model. The upper Venn diagram compares the peptoid features on the array that displayed intensities at least 10-fold above background from data sets taken from diseased mice at different stages of the disease (stages proceed form 0 (no symptoms) to 6 (dead)). 2076 peptoids were identified that captured high levels of antibody at all stages of the disease. Peptoids were identified that displayed intensities >10× above background in any of the data sets from the control mice and asked how many of these were also bright in the diseased data sets. As shown in the lower Venn diagram, 71 peptoids were consitently bright in all of the diseased samples and dark in all of the control samples. These 71 peptoids are therefore candidates for capture agents for disease-amplified antibodies.

FIG. 7 illustrates an evaluation of the specificity of the putative autoantibody-binding peptoids. The same experiment described in FIG. 6 for EAE mice were conducted using a lupus model. The same type of analysis identified 99 peptoids that were always bright in the lupus mice and dark in the control mice. Comparison of these 99 peptoids with the 71 identified in the EAE (MS) experiment showed that all but three were unique. These data argue strongly that this methodology is capable of identifying peptoids that capture antibodies that are amplified in a disease-sepcific fashion.

FIG. 8 shows a schematic view of how protein profiling is done using a synthetic molecule microarray. Black dots represent spotted, covalently linked compounds. Green bars represent the intensities of a fluorescent signal visualized by hybridizing with a fluorescently labeled antibody. This experiment is carried out in the context of a complex biological solution such as serum, blood, CSF, etc. and the binding pattern of the protein(s) of interest is monitored by subsequent hybridization with a labeled antibody that recognizes the protein(s) of interest. Alternatively, the primary antibody could be unlabeled and the pattern could be detected by a second hybridization with a labeled anti-IgG secondary antibody.

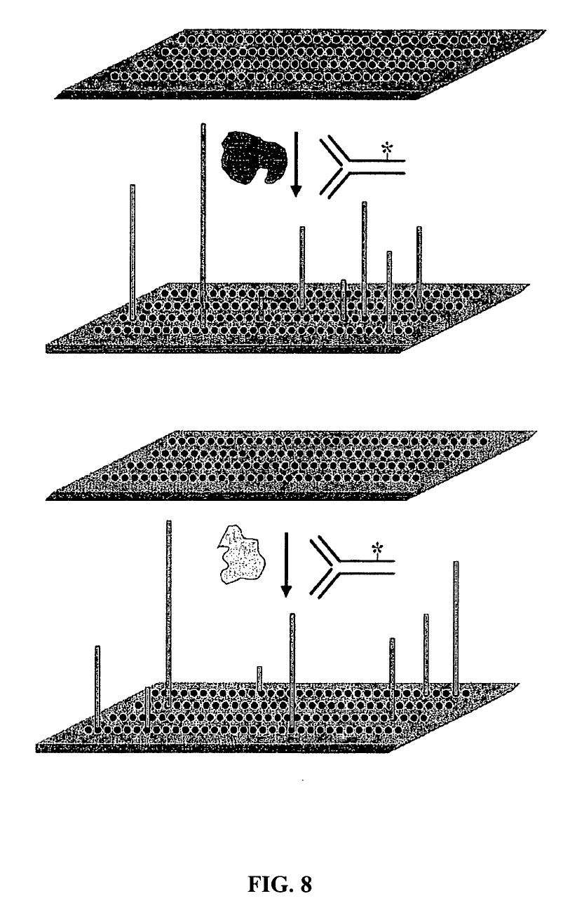

### DETAILED DESCRIPTION OF THE INVENTION

A seminal problem in biology and medicine is the discovery of new biomarkers, which are molecules or cells that are reliable indicators of a particular physiological state of an organism, for example, whether or not a ptient has a particular disease. The present invention includes compositions and methods for detecting and/or discovering biomarkers. Biomarkers may be present in readily available biological fluids, such as serum, by profiling the binding pattern of a given family of proteins or other type of molecule in the sample on to a large collection of unselected synthetic ligands displayed on the surface of an array. Visualization of the binding patterns and comparison of those obtained for two sets of samples, for example serum from patients with a disease and serum from heathy individuals, serves to identify molecules on the array that bind to ligand binding moieties, which wil include biomarkers, whose levels are significantly increased or decreased one set of samples relative to the other.

Embodiments of the invention include methods and compositions for the discovery of ligand binding moieties and synthetic compounds (i.e., ligands) that capture such from a complex sample. These samples can contain a complex mixture of components, such as, but not limited to, proteins, peptides, lipids, carbohydrates, small molecules, or cells. In certain aspects of the invention, the binding array is referred to as a random array due to the fact that the structure, composition, and/or organization of the binding elements are not designed or pre-selected to bind any particular component of a sample. That is, the initial design of the array is not biased. However, the compounds displayed on the array, referred to as syntheitic molecules, ligands, or binding elements, may not be completely random in the structural and molecular sense, in that they may all share certain chemical features, for example all being members of a particular class of compounds, such as peptoids. The identity of the binding elements may be known or characterized subsequently if one wishes, and supplementary arrays may then be made that take this binding activity into account (these are called “biased” or “focused” arrays). Moreover, the process for making the array is reproducible in the sense that each array contains the same chemical element in the same position on the array, allowing comparison of the binding of potential biomarkers to two or more different arrays. The binding profile of a component in a sample to the array will be used in assessing or detecting differences in the sample as compared to a standard or second sample.

Components of the sample will bind each binding element of the array with various affinities and specificities. The binding affinity and specificity between most of the binding elements and a sample component will typically be insufficient for detection of the complex above background or a particular signal threshold above background. A subset of binding elements, however, will bind a sample component with sufficient affinity and specificity for the complex to be detected. Aspects of the invention include sufficient binding of two or more elements of an array, to a component of the sample mixture at levels detectable above a certain threshold. The exposure of an array to a sample results in a unique pattern of binding for a given sample component, class of sample components, subset of sample components, or a group of target components present in a sample. Methods may further include assessing binding of a control or known molecule to a ligand or array. The resulting binding profile, fingerprint, or signature can be likened to a “topographical binding profile” for components of a mixture. Samples derived or obtained from sources having different characteristics will display different binding profiles for one or more sample components and the subset of binding elements that reveal prominent differences between the samples can be used to construct focused arrays capable of reliably distinguishing between the physiological states of interest (for example, a disease state and a healthy state). Embodiments of the invention may also include biasing the otherwise random collection of synthetic binding elements by including in most or all of them a chemical fragment known or suspected to facilitate binding to the class of potential biomarkers of interest. Such chemical fragments can include an inhibitor or modulator of a component or class of components or analogues of such inhibitors or modulators.

A binding profile or signature can be predictive of a condition or disease state in a subject from which the sample was obtained, including binding profiles associated with isoforms and derivatives of ligand binding moieties, for example post-translationally modified forms of a protein. Such binding profiles can be indicative of the activation, inactivation, and/or modulation of a cellular pathway, such as signal transduction pathways. Aspects of the invention include stimulation of a sample or sample source prior to obtaining a sample or prior to contacting the sample with an array. Stimulation includes contacting a sample (e.g., serum) or sample source (e.g., a patient) with a drug, a protein, an enzyme, a therapy or a therapeutic regime, a diet, or maintaining such in a particular environment or under a particular set of conditions (e.g., oxidative stress). Further aspects include stimulating a sample (e.g., cells, biopsy, etc.) in vitro, in situ, or in vivo. An environment or set of conditions can include, but are not limited to, conditions related to light, heat, cold, sleep deprivation, fasting, elevated noise, sound deprivation, light deprivation, and the like. Typically, a subset of the components of a sample will correlate to a particular disease or condition, such as an autoimmune disease state or a particular form of cancer. In other embodiments, a binding profile may be chosen to detect the presence or absence of one or more pathogen, such as fungi, bacteria, viruses, parasites or a portion or by product thereof.

Sample components include a variety of ligand binding moieties such as proteins, which include, but are not limited to antibodies, serum proteins, enzymes, cytokines, cell surface receptors, intracellular signaling proteins, chaperones, structural proteins, etc. A sample can include, but is not limited to, an environmental or a biological sample, such as water, soil, air, culture, serum, blood (including whole blood or portions thereof), cerebrospinal fluid, sputum, semen, and/or saliva samples. Sample can be obtained from environmental sites or from animals, including but not limited to animal subjects, such as cows, pigs, horses, birds, chickens etc. and human subjects.

The profiling technique may be used in the assessment of a complex mixture such as a serum sample, a biopsy, or a cell or tissue extract. Purification or partial purification of one or more sample components need not be, but may be, performed prior to assessment using the present methods and compositions. Components of the sample can be bound to or associated with a binding element array and particular compounds or classes of compounds may then be selectively assessed or detected. Selective assessment can be performed, for example, using various immunoassays, which are well know to those in this field (e.g., ELISA and sandwich assays using antibodies that are specific for a protein or class of proteins) or various biophysical techniques (e.g., mass spectrometry). The signal inherent to the assessment means can be determined and designated as “background.” Typically, the background will be assessed and subtracted from the signal calculated or generated as representative of detecting binding to the array. The background determination may also be used as a base for establishing a threshold for selecting signal levels/binding to included in the binding profile.

As referred to above, one aspect of the invention is to measure many different components of the same class of components simultaneously, for example assessing an antibody profile. All antibodies are quite similar, but have divergent antigen binding sites. Thus, any particular antibody would be expected to provide a pattern that is unique, though there would be some overlap between the patterns. The binding of any antibody to the array could be visualized by using a class-specific detection reagent, for example a labeled anti-IgG secondary antibody. When a complex sample such as serum is exposed to the array, the binding “superpattern” visualized will be comprised of the sum of each of the individual antibody binding patterns weighted by their abundance in the sample. This “superpattern” would therefore be indicative of a disease state or physiological state because it would reflect the production of antibodies not present in a healthy state or a different physiological state, since it is reasonable to assume that the immune, or other biological system(s) of an individual will react to a variety of conditions or maladies (e.g., cancer, infectious disease, atherosclerosis, autoimmune disease, sleep disorders, etc.) in a unique way. Given a sufficient number of retrospective studies to couple particular binding profiles or signatures with conditions and/or disease states, the same diagnostic protocol could be employed to detect (clinically and pre-clinically) a large variety of medical conditions using random arrays or focused arrays derived from studies using the random arrays.

Another example of this type of measurement would be to expose a complex mixture such as a cellular extract to the array and measure the superpattern formed by all phosphotyrosine-contianing proteins by subsequent exposure of the array to an anti-phosphotyrosine antibody. In general, any class of proteins for which there exists an antibody or other binding agent that recognizes most or all members of that class of proteins could be profiled in this manner.

In another aspect, the structures of the ligands displayed on the array could be biased somewhat to encourage binding of a given class of proteins to them. For example, an ATP analogue could be coupled to a collection of otherwise random molecules to increase the general affinity of these molecules for ATP-binding proteins. In general, an array of otherwise random compounds could be biased to bind a family of co-factor-binding protein by appending the cofactor or a mimic of it to each compound displayed on the array.

The methods of assessment will be modified as needed to compensate for the variation in binding profiles or signatures between different individuals or samples, so that a profile from a given subject or sample is indicative of a condition or state, such as developing cancer. Also, the specificity of a binding profile or signature for particular conditions can be assessed to differentiate or compensate for two or more conditions that have an overlapping binding profile or signature (e.g., infections). For example, providing a distinction (staphylococcus infection) or a general assessment (e.g., infection) of a sample associated with a bacterial as compared to a viral infection. It is contemplated that different infections will produce somewhat different binding profiles. Embodiments of the invention allow for binding assessments to be made in complex solutions relevant to diagnosis in medicine or other fields.

Derivatives, modifications, or conformers (collectively termed isoforms) can be detected and compared by the inventive methods. It is anticipated that different binding patterns on an array will be observed for different forms or isoforms of a component. Isoforms will essentially behave as a chemically distinct species that will exhibit a characteristic binding profile. Different forms of sample component might result from: 1) post-translational modifications, such as phosphorylation, ubiquitylation, glycosylation, or nitration; 2) alternative processing, such splicing of the mRNA splice variants and isoforms, or altered metabolons (sequential metabolic transformations); 3) proteolysis of a pre-protein (such as in the maturation of pro-hormones); 4) ligand binding (other than to the array), which for example will alter the structure of a component, such as the secondary, tertiary or quaternary structure of a protein. One may distinguish different forms of a component by assessment or detection of different binding profiles using a one or more detection schemes. In certain aspects, only a single general detection scheme is needed in contrast with multiple isoform specific detection schemes used currently, such as protein specific, conformation specific, or phosphorylation specific antibodies. The detection of binding or binding profile of the one or more isoforms detects or identifies one or more isoforms in the sample. As mentioned above, isoforms include, but are not limited to phosphorylation isoforms, glycosylation isoforms, myristoylation isoforms, ubiquitylation isoforms, oxidatively modified isoforms, SUMOlyation isoforms, notrosylation isoforms, sulfonation isoforms, length isoforms (e.g., cleavage products), amino acid substitution isoforms and/or protein conformation isoforms (e.g., prion and infective prion isoforms). In certain aspects, one or more isoforms or derivatives include, but are not limited to proteins and particularly enzymes, such as kinases and/or kinase targets. Aspects of the invention include assessing binding of one or more isoform of one or more sample components to 1, 2, 3, 4, 5, 6, 7, 8, 9, 10 or more ligands or arrays.

Two of the significant applications of the compositions and methods include diagnosis of autoimmune disease and cancer. The profiling of antibodies or other immune-associated components such as non-adherent T cells can be used to diagnose the presence of, or risk of developing, a particular condition. An additional advantage of the present invention is the cost effectiveness and usability of the methods. Finally, the present invention eliminates the major barrier to immunoprofiling. One need not have have any knowledge of the native antigens that result in the production of disease-specific antibodies or T cells. This requirement has greatly limited the production of arrays capable of detecting disease-sepcific antibodies, since the current state of the art is to use these antigens as ligands displayed on the array.

Furthermore, the methods may include assessing the state of signal transduction cascades (e.g., their activation, inactivation, or modulation). This type of embodiment can be used to assess the response of one or more subjects to drugs, particularly those in clinical trials, or development of resistance to drugs, particularly those included in standard therapies. Such methods will include obtaining a sample from a subject, e.g., cells from a patient. The sample is processed and brought in contact (hybridized) to an array, then submitted to a detection procedure or process, e.g., probed with labeled antibodies against a protein kinase or class of protein kinases involved in pathway of interest. The idea is that the binding pattern (profile) of the kinase(s) would be different depending on whether or not the kinases(s) had been activated, since this event involves post-translational modification of the protein(s), including phosphorylation. This would obviate the requirement for one or more phosphospecific antibody, which may or may not be obtainable.

A. Binding Elements

Binding elements are molecules or portions of molecules that demonstrate an affinity for a particular target, sample component, or ligand binding moiety, each term may be used interchangeably. Binding elements typically comprise peptides, peptoids, oligonucleotides, oligosaccharides, or other small molecules that are able to be produced combinatorially or by other synthetic or recombinant means. In certain aspects, the binding elements are random binding elements, at least initially. Binding elements may be selected based on known reactivity to one or more sample component or ligand binding moiety and used to produce a supplementary of secondary array that is directed to one or more sample assessment purposes. In certain embodiments, the binding elements may be preselected as a general class of elements or for specific binding affinities for an initial/primary array or for a supplementary/secondary array.

Binding elements are typically operatively coupled to a support as described herein. “Low affinity,” as used herein, is defined as an interaction with a dissociation constant (K_{D}) of ≧10⁻⁵ M, “moderate affinity” as used herein is defined as a K_{D} between 10⁻⁵ M and 10⁻⁸ M, and “high affinity” as used herein is defined as a K_{D} of ≦10⁻⁸ M. Binding elements may be based on a variety of molecules or substances. In various embodiments a binding element(s) may include, but is not limited to, a peptide, a peptoid (i.e., N-substituted oligoglycines), a peptide-like molecule, a polypeptide, a oligosaccharide, a nucleic acid, a small molecule, an inorganic molecule, an organic molecule or the like. It is also contemplated that combinations of different classes of binding elements may also be used, for example, a peptide modified with a small molecule and the like. It is contemplated that combinations of different classes of binding elements may be used in forming a chimeric binding element, for example, a peptoid with a small molecule as a capping molecule (ATP or an ATP analog) and the like. Thus, a ligand may be wholly random, partially random, biased or non-biased In some embodiments, binding elements may be covalently coupled or fused to each other, for example a fusion of two peptides, with or without intervening residues, into a single linear molecule, i.e., a chimeric binding element. For each binding element, a preferred density may be empirically determined by arraying a number of sensing elements (sudivisions of an array), which include one or more binding elements, at varying densities and identifying an optimal binding element density.

One or more different types of binding elements can be immobilized on a support surface. Binding elements may be localized or segregated to particular regions on a support or on particular supports, e.g., latex beads. Each of these particular regions will be able to bind at least one target or sample component. These regions are referred to as sensing elements or regions. Typically, at least 2, 3, 4, 5, 6, 7, 8, 9, 10, 20, 30, 40, 50, 100, 200, 300, 400, 500, 600, 700, 800, 900, 1000, 5000, 10,000, 100,000 or more different sensing elements (including all values and ranges therebetween), can be immobilized on a support surface to form various arrays.

In certain aspects, binding elements may be identified or preselected so that a number of binding elements are associated with components of a target pathway, disease, or organism. Having a number of elements that bind to proteins or other molecules involved in various pathways, diseases, or organisms on a support allows those skilled in the art to readily determine which component in a sample is, for example present, defective, and/or over expressed in a sample for multiple disease states or conditions at the same time. In some embodiments, a sample may be related to normal/non-normal cell development, normal/disease condition, infected/non-infected condition, presence/absence of an organism/agent and the like. Smart arrays may have a subset(s) of the array having one or more binding element that is indicative of a disease or condition. A subset of the array may be associated with a particular subsection of the array (e.g., columns, rows or subarrays). The smart array is selected and organized based on results from random arrays and may address a plurality of related and unrelated conditions. Each condition addressed will be in register with a particular subsection of the smart array.

1. Small Molecules

Virtually any molecule or compound having an ability to bind a target molecule may be used as a binding element. Binding elements may include non-biological or biological polymers, oligosaccharides, a variety of small molecules, lipids, and the like.

Methods have been developed for the combinatorial (e.g., rapid-serial or parallel) synthesis and screening of libraries of small molecules of pharmaceutical interest, and of biological oligomers such as peptoids, polypeptides, proteins, oligonucleotides and deoxyribonucleic acid (DNA) polymers (Eichler et al., 1995; Cho et al., 1999; LePlae et al., 2002; Ostergaard and Holm, 1997; Yang et al., 1999). U.S. Patents 6,475,391 and 6,461,515; and Brocchini et al. describe exemplary methods and compositions for the preparation and characterization of polymer combinatorial libraries for selecting polymer materials (Brocchini et al., 1997). Exemplary synthetic methods for oligosaccharides is provided in Kanemitsu and Kanie (2002).

Various small molecule libraries may be obtained from commercial or non-commercial sources, as well as synthesizing such compounds using standard chemical synthesis technology or combinatorial synthesis technology (see U.S. Pat. No. 6,344,334; Gallop et al., 1994; Gordon et al., 1994; Thompson and Ellman, 1996; each of which is incorporated herein by reference).

2. Peptides and Peptide-like Molecules

In various aspects of the invention, peptides, peptoids, polypeptides, and/or proteins may be used as a binding element or as a portion of an array. The peptides, polypeptides and/or proteins used as a binding element may be an isolated, a recombinant, or a synthetic peptide(s), peptoid(s), polypeptide(s), proteins, oligomeric molecule, and/or small molecule. Typically, the composition of a peptide, peptoid, polypeptide or other oligomer will be variable.

3. Synthetic Peptides

Various embodiments of the invention describe peptides or peptide mimetics for use in the production of binding elements. Peptides, peptide mimetics or peptide like molecules of the invention may also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide or oligomeric sequences, or libraries of overlapping peptides or oligomers, usually from about 6 up to about 35 to 50 amino acids or monomers correspond to binding elements described herein, can be readily synthesized and then screened in screening assays designed to identify binding profiles of interest. In some embodiments, recombinant DNA technology may be employed wherein a nucleotide sequence which encodes a peptide of the invention is inserted into an expression vector, transformed or transfected into an appropriate host cell and cultivated under conditions suitable for expression.

4. Fusion Peptides

A specialized kind of insertional variant is the fusion protein or peptide. This molecule generally has all, a substantial portion, or a portion of a first molecule, linked at the N- or C-terminus, to all or a portion of a second molecule. For example, fusions typically employ leader sequences from other species to permit the recombinant expression of a protein in a heterologous host. Other useful fusions include linking of binding elements. Fusions of the invention include a fusion of two or more binding elements. In certain embodiments the two or more elements are reversibly or irreversibly coupled to each other.

5. Nucleic Acids

In certain embodiments, binding elements may comprise nucleic acids. As discussed below, a nucleic acid may contain a variety of different bases and yet still produce a binding element. The methods of the present invention may select and use nucleic acids that bind to a variety of substances with a low to moderate affinity.

B. Synthesis and/or Purification of Binding Elements

In certain embodiments, it may be desirable to purify or partially purify a binding element. A variety of purification techniques for a variety of compounds are well known to those of skill in the art. These techniques involve, at one level, the crude fractionation of a milieu to fractions containing and not containing a binding element. Having separated the binding element from other contaminants, the binding element may be further purified using chromatographic and electrophoretic techniques to achieve partial or complete purification (or purification to homogeneity). Analytical methods particularly suited to the preparation of a particular binding element are ion-exchange chromatography, exclusion chromatography; polyacrylamide gel electrophoresis; isoelectric focusing. A particularly efficient method of purifying binding element is fast protein liquid chromatography or even HPLC.

Certain aspects of the present invention concern the purification, and in particular embodiments, the substantial purification, of a binding element. The term “purified binding element” as used herein, is intended to refer to a composition, isolatable from other components, wherein the binding element is purified to any degree relative to its state of synthesis, production, or naturally obtainable state. A purified binding element therefore also refers to a binding element, free from the environment in which it may naturally occur. Generally, “purified” will refer to a binding element composition that has been subjected to fractionation to remove various other components, and which composition substantially retains its activity. Where the term “substantially purified” is used, this designation will refer to a composition in which the protein, peptide, or binding element forms the major component of the composition, such as constituting about 50%, about 60%, about 70%, about 80%, about 90%, about 95% or more of the binding elements in the composition. Various methods for quantifying the degree of purification will be known to those of skill in the art in light of the present disclosure.

Various techniques suitable for use in purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified binding element.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This can be a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

C. Binding Element Arrays

In various embodiments of the invention binding elements or ligands may be operatively coupled to a support. A “support” refers to a solid phase onto which a binding element can be provided, (e.g., by attachment, deposition, coupling and other known methods). One or more binding elements may be immobilized on supports including, but not limited to glass (e.g., a chemically-modified glass slide), latex, plastic, membranes, microtiter, wells, mass spectrometer plates, beads (e.g., cross-linked polymer beads) or the like. A binding element array can include, but is not limited to a plate, a chip, and/or a population of beads. A variety of array formats are known in the art and can be adapted to the inventive methods based on the descriptions provided in this application.

In one aspect, the invention provides supports adapted for use with a detector or a detection method(s) (e.g., ELISA or mass spectrometry), wherein the support comprises a binding elements immobilized on the support surface. The binding elements will typically bind with some affinity and specificity to one or more component(s) of a sample. In various non-limiting embodiments, the sample is a biological sample. The component may be involved in a biological pathway (e.g., signal transduction, immunological response, cytoplasmic or membrane enzyme mediated pathway, cell cycle or developmental cycle pathway). Typically, binding element(s) are located at different addressable, segregated regions referred to as sensing elements or regions on a support so that one can readily distinguish which components in a sample are bound to a support. In some embodiments, binding elements can be placed in the same sensing element or region of the support as long as the components can be differentially detected. The supports and the binding elements are described in detail herein.

A target(s) (i.e., a sample component or ligand binding moiety) present in a sample can be captured or bound on any of a variety of binding element array/support combinations. Exemplary protein biochips described in the art are biochips produced by Ciphergen Biosystems (Fremont, Calif.), Packard BioScience Company (Meriden Conn.), Zyomyx (Hayward, Calif.) and Phylos (Lexington, Mass.) (see for example U.S. Pat. Nos. 6,225,047 and 6,329,209, and International publication WO 99/51773 and WO 00/56934, each of which is incorporated herein by reference).

In certain embodiments of the invention, a surface may comprise a plurality of addressable locations, each of which location has one or more binding elements. The binding element can be a biological molecule, such as a peptide, polypeptide, or a nucleic acid, which binds other biomolecules in a specific manner. Binding elements can comprise a purely random feature and a non-random feature.

In one embodiment, a support is capable of being engaged by an interface of a mass spectrometer which positions the support in an interrogatable relationship with an ionization source. The support can be in any shape, e.g., in the form of a strip, a plate, or a dish with a series of wells. Each binding element(s) may be immobilized at different addressable locations at the support surface.

Typically, each sensing element or region comprises a different binding element(s) so that one can readily distinguish a binding pattern or profile of one or more targets in a sample that are bound to the support.

Each sensing region on the support will be “addressable” in that during detection of target binding, a detection method may be directed to, or “addresses” the sensing region(s) where a target is bound to the one or binding elements. The addressable locations can be arranged in any pattern on the support, but are preferably in regular pattern, such as lines, orthogonal arrays, or regular curves (e.g., circles). Alternatively, binding elements can be placed on the support surface in continuous patterns, rather than in discontinuous patterns.

Alternatively, the support can be a separate material. For example, a support can be a solid phase, such as a polymeric, paramagnetic, latex, or glass bead, upon which are immobilized binding elements for one or more targets. A solid phase material may be placed onto a probe or detectable media (e.g., fluorescently tagged bead) that is removably insertable into a gas phase ion spectrometer or passed by a detector such as a laser/spectrometer device. The solid phase with each type of binding element(s) is typically placed at different addressable locations of the support surface. Alternatively, as noted above, different binding elements can be placed on the same addressable locations as long as they are able to be differentially detected.

The support can be also shaped so that it is adapted for use with various components of a gas phase ions spectrometer, such as inlet systems and detectors. For example, the support can be adapted for mounting in a horizontally and/or vertically translatable carriage that horizontally and/or vertically moves the support to a successive position. This allows components bound to different locations of the support surface to be analyzed without requiring repositioning of the support by hand.

The support can be made of any suitable material. For example, the support materials include, but are not limited to, insulating materials (e.g., glass such as silicon oxide, plastic, ceramic), semi-conducting materials (e.g. silicon wafers), or electrically conducting materials (e.g., metals, such as nickel, brass, steel, aluminum, gold, or electrically conductive polymers), organic polymers, biopolymers, or any combination thereof. The support material can also be solid or porous. Examples of supports suitable for use in embodiments of the invention are described in U.S. Pat. No. 5,617,060 and PCT Publication WO 98/59360, each of which are incorporated by reference.

The support can be conditioned to bind binding elements. In some embodiments, the surface of the support can be conditioned (e.g., chemically or mechanically (e.g., roughening)) to place binding elements on the surface. Typically, a support comprises reactive groups that can immobilize binding elements. For example, the support can comprise a carbonyldiimidazole group which covalently reacts with amine groups. In another example, the support can comprise an epoxy surface which covalently reacts with amine and thiol groups. In another example, the support could be a glass suface in which the surface is modified by first appending a poly-ethylene glycol chain followed by capping with a thiol-reactive moiety such as a maleimide, which reacts covalently with a thiol-containg ligand. Supports with these reactive surfaces are commercially available from Ciphergen Biosystems (Fremont, Calif.) or can be synthesized using protocols known to those knowledgable in the art.

Arrays utilized in this invention may include between about 10, 100, 1,000, 2,000, 3,000, 4,000, 5,000, 6,000, 7,000, 8,000, 9,000, 10,000, 12,500 to 25,000, 50,000, 75,000, to about 100,000 distinct random ligands or binding elements, including values and ranges therebetween.

III. Sample Preparation and Handling

The components of samples that can be explored as biomarkers using this invention may be non-adherent cells (e.g., immune effector cells, such as T-cells and the like), microorganisms (e.g., pathogenic and opportunistic microbes, including bacteria, fungi, virus and the like), proteins, peptides, lipids, polysaccharides, small molecules, organic molecules, inorganic molecules, biological molecules, and the like. In particular aspects, the sample components to be evaluated as potential biomarkers are antibodies or proteins present in a sample derived from a subject (e.g., serum, biopsy, urine, CSF etc.). Samples used in this invention can be derived from a range of sources, from biological samples to environmental samples. In particular embodiments the sample may be derived from a biological source. These include, e.g., body fluids such as blood, feces, sputum, urine, serum, saliva, or extracts from biological samples, such as biopsies, bacteria or cells. Samples may be derived or obtained from a variety of subjects, including animals, both domestic and wild. Subjects include, but is not limited to humans, including patients and clinical subjects; livestock, such as cows, pigs, goats, sheep, and horses; fowl, such as chickens, ducks, guineas, and turkeys; pets, such as dogs, cats, guinea pigs, and reptiles. In certain embodiments, a sample is in liquid form. In some embodiments a sample may be derived from a gas or transformed into a gas or liquid.

Typically the sample is contacted with a support comprising an array of binding elements in any suitable manner, e.g., bathing, soaking, dipping, spraying, washing over, or pipetting. Generally, a volume of sample containing from 1 pM to 1 mM of a target in a volume from about 1 μl to 1 ml is sufficient for binding to one or more binding elements. The sample can contact the support comprising one or more binding elements for a period of time sufficient to allow the target molecules to bind to the binding element(s). Typically, the sample and the support comprising the binding elements are contacted for a period of between about 30 seconds to about, 1, 5, 10, 20, 30, 40, 50 minutes to about 1, 2, 3, 4, 5, 6, 7, 8, 9, 10, 11, 12, to 24 hours or so. In some embodiments, between about 30 seconds and about 15 minutes is sufficient for binding of the target. Typically, the sample is contacted with the binding elements under ambient temperature and pressure conditions. For some samples, however, modified temperature (typically at about 4, 5, 10, 15, 20, 25° C. to about 30, 32, 34, 36, to 37° C.) and pressure (atmospheric pressure to 1, 5, 10, 15, 20, 25, 30 or more psi) conditions may be desirable. These conditions are determinable by those skilled in the art.

After the support is contact with the sample or sample solution, it is preferred that unbound and weakly absorbed materials on the support surface are washed out or off so that only the more tightly bound materials remain on the support surface. Washing a support surface can be accomplished by, e.g., bathing, soaking, dipping, rinsing, spraying, or washing the support surface with an eluant. A microfluidics process may be used when an eluant is introduced to small spots of capture agents on the support. Typically, an eluant may be at a temperature of between less than 0, 5, 10, 15, 20, 25, 30, 35, 40, 45, 50, 60, 70, 80, 90 to 100° C. or any value or range therebetween. In some embodiments, washing unbound materials from the probe surface may not be necessary if components bound by binding elements can be resolved by gas phase ion spectrometry without a wash or are detected using a high specificity sandwich reagent that will ignore molecules that might be present other than the target.

Any suitable eluants (e.g., organic or aqueous) that preserve the relevant interaction can be used to wash the support surface. Preferably, an aqueous solution is used. Exemplary aqueous solutions include, e.g., a HEPES buffer, a Tris buffer, or a phosphate buffered saline. To increase the wash stringency of the buffers, additives can be incorporated into the buffers. These include, but are limited to, ionic interaction modifier (both ionic strength and pH), hydrophobic interaction modifier, chaotropic reagents, affinity interaction displacers. Specific examples of these additives can be found in, e.g., PCT publication WO98/59360. The selection of a particular eluant or eluant additives is dependent on the conditions used (e.g., types of binding elements used, and/or types of compounds or molecular targets, such as signal transduction components, immunological components, cell cycle or developmental cycle components, etc.).

Prior to desorption and ionization of a target from a support surface, an energy absorbing molecule (“EAM”) or a matrix material is typically applied to the support surface. The energy absorbing molecules can assist absorption of energy from an energy source from a gas phase ion spectrometer, and can assist desorption of targets from the support surface. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in the art. See, e.g., U.S. Pat. No. 5,719,060 for additional description of energy absorbing molecules.

The energy absorbing molecule and the sample can be contacted in any suitable manner. For example, an energy absorbing molecule is mixed with the sample, and the mixture is placed on the support surface. In another example, an energy absorbing molecule can be placed on the support surface prior to contacting the support surface with the sample. In another example, the sample can be placed on the support surface prior to contacting the support surface with an energy absorbing molecule. Then the components bound to the capture reagents on the support surface are desorbed, ionized and detected as described in detail below.

IV. Detection Methods

Methods detecting targets captured or bound on a solid support can generally be divided into photometric methods of detection and non-photometric methods of detection.

Photometric methods of detection include, without limitation, those methods that detect or measure absorbance, fluorescence, refractive index, polarization or light scattering. Methods involving absorbance include measuring light absorbance of an analyte directly (increased absorbance compared to background) or indirectly (measuring decreased absorbance compared to background). Measurement of ultraviolet, visible and infrared light all are known. Methods involving fluorescence also include direct and indirect fluorescent measurement. Methods involving fluorescence include, for example, fluorescent tagging in immunological methods such as ELISA or sandwich assay. Methods involving measuring refractive index include, for example, surface plasmon resonance (“SPR”), grating coupled methods (e.g., sensors uniform grating couplers, wavelength-interrogated optical sensors (“WIOS”) and chirped grating couplers), resonant mirror and interferometric techniques. Methods involving measuring polarization include, for example, ellipsometry. Light scattering methods (nephelometry) may also be used.

Non-photometric methods of detection include, without limitation, magnetic resonance imaging, gas phase ion spectrometry, atomic force microscopy and multipolar coupled resonance spectroscopy. Magnetic resonance imaging (MRI) is based on the principles of nuclear magnetic resonance (NMR), a spectroscopic technique used by scientists to obtain microscopic chemical and physical information about molecules, for a review see Hornak (2002). Gas phase ion spectrometers include mass spectrometers, ion mobility spectrometers and total ion current measuring devices.

Mass spectrometers measure a parameter which can be translated into mass-to-charge ratios of ions. Generally ions of interest bear a single charge, and mass-to-charge ratios are often simply referred to as mass. Mass spectrometers include an inlet system, an ionization source, an ion optic assembly, a mass analyzer, and a detector. Several different ionization sources have been used for desorbing and ionizing analytes from the surface of a support or biochip in a mass spectrometer. Such methodologies include laser desorption/ionization (MALDI, SELDI), fast atom bombardment, plasma desorption, and secondary ion mass spectrometers. In such mass spectrometers the inlet system comprises a support interface capable of engaging the support and positioning it in interrogatable relationship with the ionization source and concurrently in communication with the mass spectrometer, e.g., the ion optic assembly, the mass analyzer and the detector.

Solid supports for use in bioassays that have a generally planar surface for the capture of targets and adapted for facile use as supports with detection instruments are generally referred to as biochips.

In certain embodiments, methods for detecting components of a biological pathway, e.g., a signal transduction pathway, wherein the methods may comprise: providing a support comprising a plurality of binding elements immobilized on a surface of the support, wherein binding elements specifically bind to one or more target component(s) of a sample, contacting a sample with a support, and detecting the components of the biological pathway bound to their corresponding capture agents on the support by gas phase ion spectrometry. In some embodiments, data generated by gas phase ion spectrometry from a test sample can be compared to a control to determine if there is any defect in the biological pathway in the test sample. The sample preparation methods and gas phase ion spectrometry analysis are described in U.S. Patent Application 20020137106, incorporated herein by reference.

Assessment of binding can include contacting the array with labeled affinity reagent, such as an anti-Ig.

V. Analysis of Data

Data generated by quantitation of the amount of a sample component of interest bound to each binding element on the array (e.g., signal transduction components, immunological components, plasma membrane enzyme mediators, cell cycle components, developmental cycle components, or pathogen components) can be analyzed using any suitable means. In one embodiment, data is analyzed with the use of a programmable digital computer. The computer program generally contains a readable medium that stores codes. Certain code can be devoted to memory that includes the location of each feature on a support, the identity of the binding elements at that feature and the elution conditions used to wash the support surface. The computer also may contain code that receives as input, data on the strength of the signal at various addressable locations on the support. This data can indicate the number of targets detected, including the strength of the signal generated by each target.

Data analysis can include the steps of determining signal strength (e.g., height of peaks) of a target(s) detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each target can be displayed in the form of relative intensities in the scale desired. Alternatively, a standard may be admitted with the sample so that a peak from the standard can be used as a reference to calculate relative intensities of the signals observed for each target detected.

Data generated by detection of component(s) in a test sample can be compared to control data to determine if the target(s) in the test sample is normal. Control data refers to data obtained from comparable samples from a normal cell, sample, or person, which or who is known to have defined profile with regard to a sample component or a sample condition. For each component being detected, a control amount of a component from a normal or standardized sample can be determined. Preferably, the control amount of a component is determined based upon a significant number of samples taken from samples such as normal cells or persons so that it reflects variations of the amount of these targets seen in the normal cell or population.

If the test amount of a particular component is significantly increased or decreased compared to the control amount of the component, then this is a positive indication that the test sample has an underlying defect or contains a particular test substance or organism, or is diagnostic of a particular condition or disease. For example, if the test amount of a biological pathway component is increased or decreased by at least 5-fold or greater than 10-fold compared to the control amount, then this is an indication that the test sample is distinct from a standard or control sample or has an alteration in a biological or non-biological system. At least 1, 5, 10% or more of the elements, including all values and ranges there between, on the array may meet the 10 fold threshold.

Data generated by the detector, e.g., the mass spectrometer, can then be analyzed by computer software. The software can comprise code that converts signal from the detector into computer readable form. The software also can include code that applies an algorithm to the analysis of the signal to determine whether the signal represents a “peak” in the signal corresponding to a target. The software also can include code that executes an algorithm that compares signal from a test sample to a typical signal characteristic of “normal” or standard sample and determines the closeness of fit between the two signals. The software also can include code indicating whether the test sample has a normal profile of the target(s) or if it has an abnormal profile.

VI. Conditions of Disease States

A binding profile of one or more sample components (biomarkers) can be used to predict, diagnose, or assess a condition or disease state in a subject from which the sample was obtained. A disease state or condition includes, but is not limited to cancer, autoimmune disease, inflammatory disease, infectious disease, neurodegenerative disease, cardiovascular disease, bacterial infection, viral infection, fungus infection, prion infection, physiologic state, contamination state, or health in general. The methods of the invention can use binding profiles and binding element/random ligands to differentiate between different forms of a disease state, including pre-disease states or the severity of a disease state. For example, the methods may be used to determine the metastatic state of a cancer or the susceptibility to an agent or disease state. Embodiments of the invention include methods and compositions for assessing ligand binding moieties present in breast cancer, lung cancer, prostate cancer, cervical cancer, head & neck cancer, testicular cancer, ovarian cancer, skin cancer, brain cancer, pancreatic cancer, liver cancer, stomach cancer, colon cancer, rectal cancer, esophageal cancer, lymphoma, and leukemia.

Further embodiments can be used to assess ligand binding moieties present in autoimmune diseases such as myasthenia gravis, chronic active hepatitis, primary biliary cirrhosis, dilated cardiomyopathy, myocarditis, autoimmune polyendocrine syndrome type I (APS-I), autoimmune hepatitis, cystic fibrosis vasculitides, acquired hypoparathyroidism, goodpasture syndrome, Crohn disease, coronary artery disease, pemphigus foliaceus, pemphigus vulgaris, Guillain-Barré syndrome, Type 1 diabetes, stiff man syndrome, Rasmussen encephalitis, autoimmune gastritis, Addison disease, insulin hypoglycemic syndrome (Hirata disease), Type B insulin resistance, acanthosis, systemic lupus erythematosus (SLE), pernicious anemia, treatment-resistant Lyme arthritis, polyneuropathy, multiple sclerosis, demyelinating diseases, Rheumatic fever, atopic dermatitis, primary biliary cirrhosis, Graves disease, autoimmune hypothyroidism, vitiligo, thyroid associated ophthalmopathy, autoimmune thyroiditis, autoimmune Hashimoto thyroiditis, coeliac disease, ACTH deficiency, myositis, dermatomyositis, polymyositis, dermatomyositis, Sjögren syndrome, systemic sclerosis, progressive systemic sclerosis, systemic sclerosis, scleroderma, morphea, primary antiphospholipid syndrome, bullous pemphigoid, herpes gestationis, cicatricial pemphigoid, chronic idiopathic urticaria, connective tissue syndromes, necrotizing and crescentic glomerulonephritis (NCGN), systemic vasculitis, Wegener granulomatosis, Churg-Strauss syndrome, polyrnyositis, Raynaud syndrome, chronic liver disease, visceral leishmaniasis, autoimmune C1 deficiency, membrane proliferative glomerulonephritis (MPGN), prolonged coagulation time, autoimmune thrombocytopenia purpura, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome, Lambert-Eaton myasthenic syndrome, and paraneoplastic cerebellar degeneration.

Yet further embodiments of the invention include methods and compositions for assessing ligand binding moieties present in infectious diseases such as Acquired immunodeficiency syndrome (AIDS), Anthrax, Botulism, Brucellosis, Chancroid, Chlamydial infection, Cholera, Coccidioidomycosis, Cryptosporidiosis, Cyclosporiasis, Diphtheria, Ehrlichiosis, Arboviral Encephalitis, Enterohemorrhagic Escherichia coli (E. coli), Giardiasis, Gonorrhea, Haemophilus influenzae, Hansen's disease (leprosy), Hantavirus pulmonary syndrome, Hemolytic uremic syndrome, Hepatitis A, Hepatitis B, Hepatitis C, Human immunodeficiency virus (HIV), Legionellosis, Listeriosis, Lyme disease, Malaria, Measles, Meningococcal disease, Mumps, Pertussis (whooping cough), Plague, Paralytic Poliomyelitis (polio), Psittacosis (parrot fever), Q Fever, Rabies, Rocky Mountain spotted fever, Rubella, Congenital rubella syndrome, Salmonellosis, Severe acute respiratory syndrome (SARS), Shigellosis, Smallpox, Streptococcal disease (invasive Group A), Streptococcal toxic shock syndrome (STSS), Streptococcus pneumoniae, Syphilis, Tetanus, Toxic shock syndrome, Trichinosis, Tuberculosis, Tularemia, Typhoid fever, Vancomycin-Intermediate/Resistant Staphylococcus aureus, Varicella, Yellow fever, variant Creutzfeldt-Jakob disease (vCJD), Dengue fever, Ebola hemorrhagic fever, Echinococcosis (Alveolar Hydatid disease), Hendra virus infection, Human monkeypox, Influenza A H5N1 (avian influenza), Lassa fever, Marburg hemorrhagic fever, Nipah virus, O'nyong-nyong fever, Rift Valley fever, Venezuelan equine encephalitis, and West Nile virus (see U.S. Government Accounting Office publication GAO-04-877 “Disease Surveillance”).

In still yet further embodiments, the invention include methods and compositions for assessing ligand binding moieties present in neurodegenerative diseases such as stroke, hypovolemic shock, traumatic shock, reperfusion injury, multiple sclerosis, AIDS, associated dementia; neuron toxicity, Alzheimer's disease, head trauma, adult respiratory disease (ARDS), acute spinal cord injury, Huntington's disease, and Parkinson's Disease.

Signal transduction cascades operate, in part, through sequential phosphorylation events mediated by protein kinases. These covalent events are critical in transducing signals from the outside of the cell to the nucleus, where they bring about changes in gene expression. The inventors claim that activation (i.e., phosphorylation) of a specific protein kinase in any specific transduction pathway could be analyzed by hybridization of a cell extract to a synthetic molecule microarray. The idea is that a chemically modified protein would evince a pattern of binding to the array distinct from that of the unmodified protein. The patterns of interest could be visualized by subsequent hybridization of the array with a labeled antibody (or an unlabeled antibody and a labeled secondary) that did not distinguish between the different forms of the protein kinase. This would remove the requirement for phospho-form-specific antibodies, which is a major technical hurdle currently in mapping signal transduction cascades. Note that this does not require the subsequent analysis of proteins or peptides bound to each feature by mass spectrometry or any other tool and does not require the identification in the mass spectrum of peaks corresponding to phosphorylated or otherwise modified peptides.

VII. Kits

Any of the compositions described herein may be comprised in a kit. In a non-limiting example, binding elements, binding element arrays and related support(s), buffers, linkers, and reagents are provided in a kit. The kit may further comprise reagents for processing a sample and/or sample components. The kit may also comprise reagents that may be used to label varous components of an array or sample, with for example, radio isotopes or fluorophors.

Kits for implementing methods of the invention described herein are specifically contemplated. In some embodiments, there are kits for synthesis, processing, and detection of binding element arrays.

Regents for the detection of sample component binding can comprise one or more of the following: array substrate; binding elements; and/or detection reagents.

The components of the kits may be packaged either in aqueous media or in lyophilized form. The container means of the kits will generally include at least one vial, test tube, plate, flask, bottle, array substrate, syringe or other container means, into which a component may be placed, and preferably, suitably attached. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing binding elements or reagents for synthesizing such, and any other reagent containers in close confinement for commercial sale. Such containers may include injection or blow molded plastic containers into which the desired vials are retained.

When components of the kit are provided in one and/or more liquid solutions, the liquid solution is typically an aqueous solution that is sterile and proteinase free. In some cases proteinatious compositions may be lyophilized to prevent degradation and/or the kit or components thereof may be stored at a low temperature (i.e., less than about 4° C.). When reagents and/or components are provided as a dry powder and/or tablets, the powder can be reconstituted by the addition of a suitable solvent. It is envisioned that the solvent may also be provided in another container means.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Sample Profiling Using Peptoid Arrays

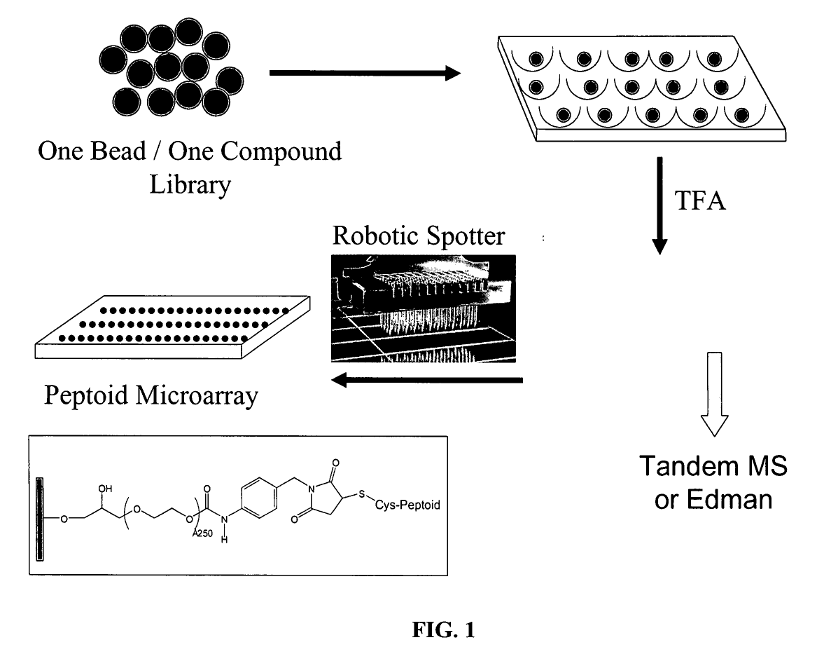

Different proteins exhibit unique and reproducible fingerprints, profiles, or signatures when hybridized to a peptoid microarray. Microarrays were constructed that consist of 7680 different octameric peptoids spotted covalently on a maleimide-functionalized glass microscope slide using a robotic pin spotter (FIG. 1). The peptoid library was created by split and pool synthesis on 500 μm polystyrene macrobeads (Rapp Polymere) using seven different amines. A C-terminal cysteine residue was included in each molecule to facilitate coupling to the array surface. These methods are described in Reddy & Kodadek (2005).

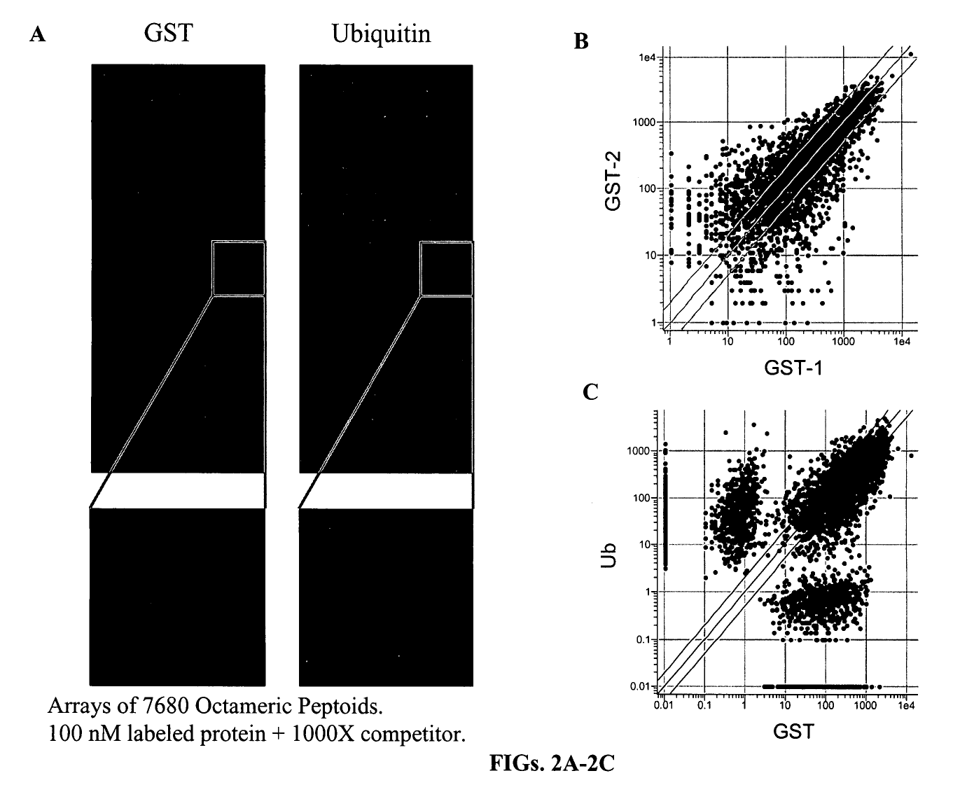

To these arrays was hybridized either fluorescein-labeled ubiquitin (Ub), fluorescein-labeled Glutathione-S-Transferase (GST) or Cy3-labeled Maltose-Binding Protein (MBP) in the presence of a 100-fold excess of unlabeled proteins derived from a crude Eschericia coli extract (to mimic a moderately abundant protein in a crude extract). GST solutions of 10, 100, or 500 nm have been used to assess the effect of dilution. The concentration of the labeled protein was 500 nM in each case. After washing, the pattern of binding of the labeled protein to the array was visualized using a standard commercial microarray scanner. Each experiment was done twice in a completely independent fashion. The raw array images from the hybridizations of labeled GST and Ubiquitin are shown in FIG. 2, along with an image of an array taken prior to. Hundreds of features on each array captured labeled protein at a level detectable above the background under these conditions, as can be better seen in the expanded regions shown in FIG. 2. These data confirm that a library of 7680 peptoids is sufficiently rich in protein ligands to support a fingerprinting application. One can fingerprint proteins using an array with less than 100 to 75 features if one so desired.

Even by the naked eye, it can be seen that the binding patterns are distinct on each array (FIG. 2). To address the issue of reproducibility and the uniqueness of the fingerprint more rigorously, all of the data were visualized as a series of scatter plots. When the two independent hybridizations for a given protein were compared, a high degree of correlation was obtained (FIG. 2 R=0.97 for GST). An even better correlation was obtained if only the higher intensity features were considered, as one would expect, since the data closest to the background tend to be the “noisiest” (not shown). These data indicate that the exemplary methods are sufficiently reproducible to serve as a platform for protein fingerprinting.

When data sets obtained from two different protein hybridizations were compared, the correlation was far lower (see FIG. 2, lower plot; R=0.56 for GST vs. Ub,). Based on these data, it was concluded that the different proteins employed in this study exhibited highly reproducible and distinctive patterns when hybridized to a peptoid microarray. In one study the average intensity difference between two studies was 5.2 fold (not shown).

### Example 2

### Distinguishing Two Antibodies by Their Binding Profile

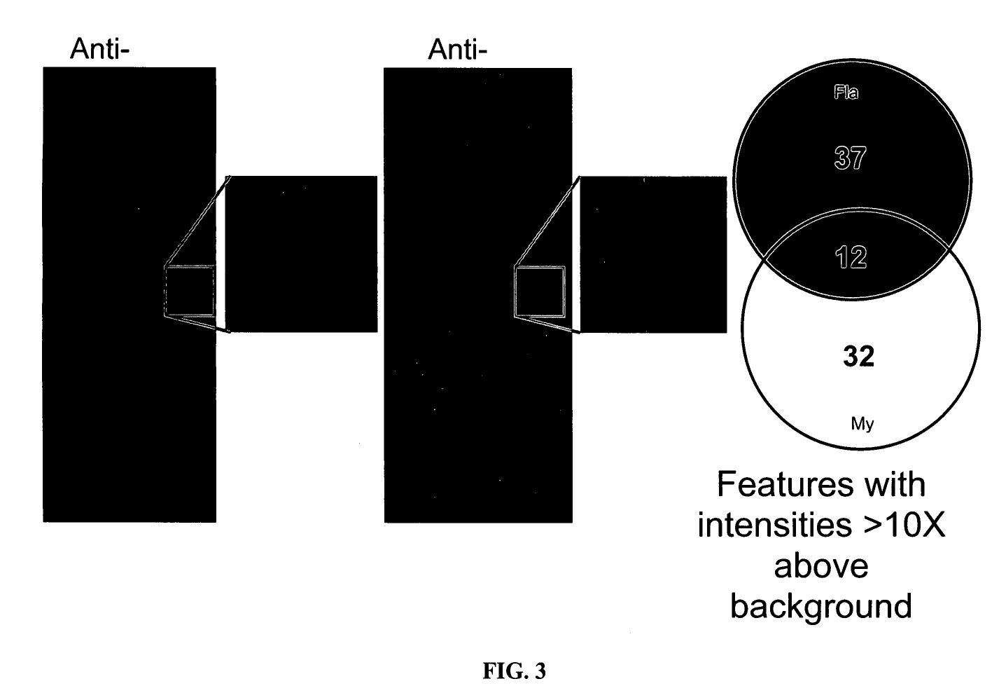

Even two proteins that are highly related chemically and structurally can be distinguished by their binding pattern to peptoid micorarrays. For example, FIG. 3 shows that two monoclonal antibodies that differ only by a few amino acids in their epitope-binding regions show clearly distinguishable binding patterns to the arrays. The binding patterns were detected by subseuqnet incubation of the arrays with a fluorescently-labeled anti-IgG antibody. A control experiment was determined to identify peptoids that bind the secondary antibody directly and these were eliminated form the analysis of the primary antibody binding patterns.

### Example 3

### Antibody Profiling in Serum

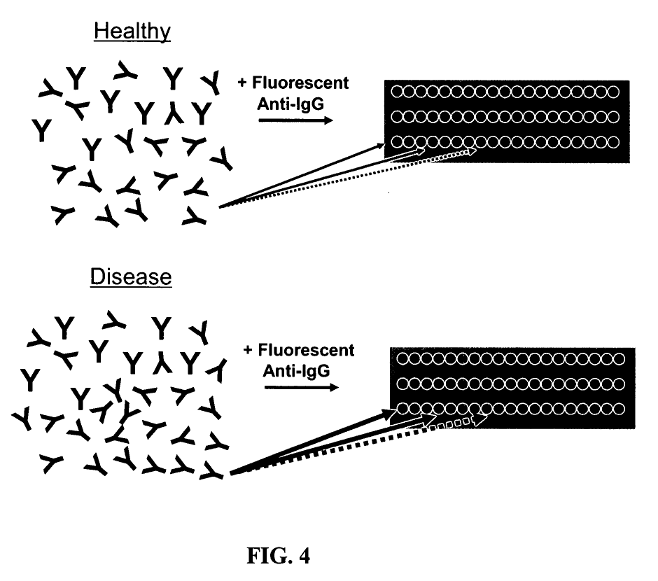

The discovery that individual monoclonal antibodies evince different binding patterns to the arrays suggested that dufferent “superpatterns” would be observed if a complex mixture of antibodies, such as that present in serum, were applied to the array. FIG. 4 illustrates the idea that this “superpattern” would be comprised of the sum of all of the individual antibody binding patterns weighted by their representation in the population. Thus, if the level of one or more antibodies were significantly elevated in a particular disease state, the intensity of signal measured at the peptoid eatures to which the amplified antibodies bind would be expected to increase detectably, thus providing a “fingerprint” specific for that disease.

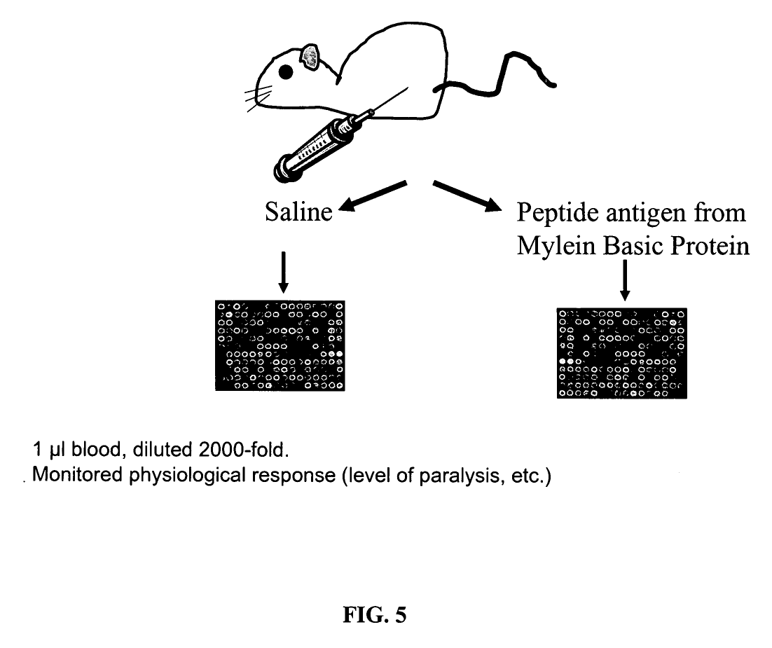

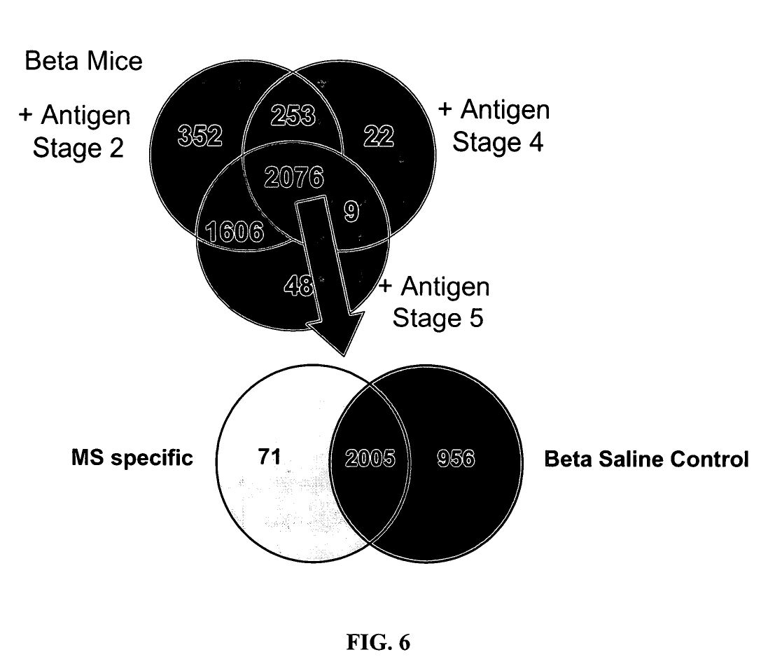

To test this idea, mice were injected with an antigenic peptide derived from MBP that is known to result in a multiples sclerosis-like autoimmune disease called EAE. As a control, some mice were injected with saline. At various times after injection, serum samples were taken from these mice and, ater dilution, were applied to peptoid microarrays and the pattern of binding of all IgG antibodies was visualized by subsequent incubation with a labeled anti-IgG secondary antibody (FIG. 5). The results, summarized in the form of a Venn diagram, show that as the disease progresses in the antigen-immunized mice, the pattern of IgG binding to the array changes. To mine these data for biomarker discovery, the peptoid features that displayed consistently high intensity in most or all of the disease samples were identified (FIG. 6; 2076 features) and compared to the peptoid features that displayed high intensity in any of the samples obtained from the saline-treated mice. As shown in the bottom diagram of FIG. 5, a comparison of these peptoids revealed that 71 consistently showed high signal intensity in most or all of the disease samples, but not in the control samples. These 71 peptoids are therefore candidates for capture agents for autoanitbodies associated with the disease state.

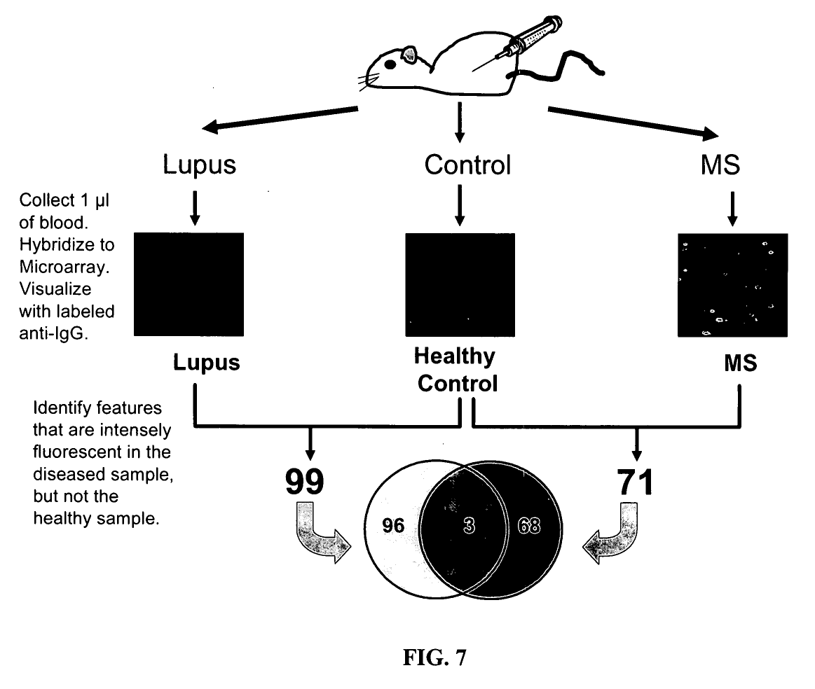

To determine if these peptoids are capturing antibodies specific to EAE, or if they represent some non-specific biological response such as inflammation, the same type of experiment was repeated with a mouse model for lupus, a different autoimmune disease. The same type of data analysis showed that 99 peptoids on the array consistently had displayed much higher levels of antibody capture in the lupus mouse-derived samples compared to the controls. As shown in FIG. 7, a comparison of the identity of the peptoids identified in the EAE (MS) and lupus studies revealed that all but three of them are unique. These data argue strongly that the inventive method is capable of identifying peptoids that capture disease-specific autoantibodies and that this approach would likely be useful for the diagnosis of human diseases to which the immune system reacts. Note that this constitutes a vey sensitive method, as the crude serum samples employed in these analyses were diluted 2000-fold prior to exposure to the array.

### Example 4

### Application to the Diagnosis of Human Disease

To assess whether the present invention might be of utility for profiling human antibody populations and might be employed to diagnose disease states, studies similar to those described in Example 3 were performed, but with serum samples collected from human patients as well as healthy controls. In each case, the serum sample from the patient with the given disease (see first column in the table below) was exposed to a microarray displaying 7680 different octameric peptoids. The resultant IgG antibody binding pattern was visualized by subsequent hybridization with a fluorescently labeled anti-IgG antibody. The IgG binding patterns of the patient sample was compared with that obtained from analysis of an age-, sex- and race-matched control subject. The results for a variety of human disease states are shown in the table below. In all of the cases except one (narcolepsy) at least one dozen (and usually far more) peptoids were identified that exhibited at least a five-fold greater signal intensisty in the diseases sample than in the control sample. Even in the case of narcolepsy, four discriminating peptoids were identified. Although larger numbers of samples must be analyzed to reach a statistically significant result, these data clearly show that human antibody profiles can be measured using peptoid microarray technology and suggest that this technique will be useful for human diagnosis of a variety of diseases. Note that the serum samples analyzed came from patients not only with an autoimmune condition (lupus, MS and rheumatoid arthritis), but cancers (Von Hippel-Landau, breast cancer, colon cancer), neurological disease (Alzheimers), infectious disease (HIV) and cardiovascular disease (heart failure).

TABLE 1
Profiling human antibody populations associated with a diagnosed
disease state.

| | Disease Signal | Disease/ | Disease/ |
|---|---|---|---|
| | >1000 & | Normal | Normal |
| Disease | Normal Signal <1000 | >5 Fold | <5 Fold |
| HIV | 675 | 68 | 607 |
| Alzheimers | 1656 | 33 | 1623 |
| Heart Failure | 1225 | 445 | 780 |
| Narcolepsy | 7 | 4 | 3 |
| Breast Cancer | 1620 | 73 | 1547 |
| Colon Cancer | 2509 | 100 | 2409 |
| Von Hippel | 3041 | 102 | 2939 |
| Lindau | | | |
| Multiple Sclerosis | 475 | 344 | 131 |
| Rheumatoid Arthritis | 26 | 14 | 12 |
| Lupus | 146 | 130 | 16 |

### Example 5

### Detection of an Immune Response Resulting from Chemical Exposure

To determine if antibody profiling on peptoid microarrays could be used to detect immune responses to exposure to toxic chemicals, rats were treated with the chemicals listed in the Table 2. An antibody profiling study was done on serum collected from these animals. As shown in Table 1, comparison of these patterns with those obtained from serum of saline-injected mice suggested that a small number of peptoids might be capable of capturing antibodies induced specifically by the particular toxic chemical. However, the differential between these serum samples was much smaller than that observed in mice and humans for the disease states discussed above. The data shown in Table 2 indicate that rat immune signatures can be obtained using this technology and indicates that it is possible to use this technique to monitor exposure to toxic chemicals.

TABLE 2
Antibody profiling of responses to exposure to toxic chemicals.

| | Treated Signal | | |
|---|---|---|---|
| | >1000 & | Treated/ | |
| | Normal Signal | Normal | Treated/Normal |
| Chemical Agent | <1000 | >2 Fold | <2 Fold |
| 1,5-Napthalenediamine | 11 | 7 | 4 |
| Benzofuran | 8 | 5 | 3 |
| N-(1- | 5 | 0 | 5 |
| naphthyl)ethylenediamine | | | |
| Pentachloronitrobenzene 2 | 6 | 3 | 3 |

### Example 6

### Mapping Cellular Responses to Drug Treatment

Signal transduction cascades operate, in part, through sequential phosphorylation events mediated by protein kinases. These covalent events are critical in transducing signals from the outside of the cell to the nucleus, where they bring about changes in gene expression. Activation (i.e., phosphorylation) of a specific protein kinase in any specific transduction pathway can be analyzed by hybridization of a cell extract to a synthetic molecule microarray. The idea is that a chemically modified protein would evince a pattern of binding to the array distinct from that of the unmodified protein. The patterns of interest could be visualized by subsequent hybridization of the array with a labeled antibody (or an unlabeled antibody and a labeled secondary) that did not distinguish between the different forms of the protein kinase. This would remove the requirement for phospho-form-specific antibodies, which is a major technical hurdle currently in mapping signal transduction cascades. Note that this does not require the subsequent analysis of proteins or peptides bound to each feature by mass spectrometry or any other tool and does not require the identification in the mass spectrum of peaks corresponding to phosphorylated or otherwise modified peptides.

To test this idea, extracts from cells that had or had not been treated with MCSF were exposed to the peptoid microarray and binding of the Akt signal transduction factor, which is known to be activated by MCSF, was assessed using a fluorescently labeled anti-Akt antibody. Analysis of the data revealed that 237 peptoids on the array were capable of distinguishing the activated and unactivatted states of Akt (which represent differential post-translational modification) by virtue of their differences in signal intensity.

### Example 7

### General Methods

Preparation of Peptoid and Peptide Microarrays. Chemicals and solvents were purchased from commercial suppliers. The combinatorial libraries used in these studies were synthesized using the “submonomer” method which employs microwave irradiation. Individual library compounds (on beads) were separated into 96 well plates (one bead per one well). These compounds were cleaved with a cocktail of trifluoroacetic acid (TFA), dichloroethane (DCE), water (H₂O) and triisopropyl silane (TIS) in the ratio of 30:65:2.5:2.5. The compounds were subsequently transferred using a robotic Tecan Genesis™ workstation to 384 well plates in a transfer buffer containing acetonitrile (ACN) and water in the ratio of 50:50. The transfer buffer was allowed to evaporate and the compounds in the 384 well plates were resuspended in DMSO. The compounds were deposited onto maleimide functionalized glass slides using a Telechem Nanoprint™ 60 microarray printing instrument. Following printing, the slides then were allowed to stand for 15 h on the printer platform, washed 1 hour each with DMSO, dimethylformamide, tetrahydrofuran, and isopropanol, dried by centrifugation and stored under argon at room temperature.

Microarray Hybridization and Image Analysis. Microarrays were first hybridized with a solution containing 1 ul of sera diluted with 999 ul of 1x TBST. Hybridization proceeded for 18 hours at 4° C. with gentle rotation. Following this hybridization, the microarrays were rinsed three times in 1x TBST (50 mM Tris, 150 mM NaCl, 0.1% Tween 20, pH 7.4) and then a second hybridization was performed for 2 hours at 4° C. using a labeled secondary antibody diluted 1:400 in 1x TBST. The slides were then washed three times in 1x TBST and dried by centrifugation.

The microarray slides were scanned by using a Molecular Devices GenePix 4200A1™ autoloading scanner at 10 micron resolution with appropriate excitation laser wavelengths. To determine the signal intensities of individual spots, the scanned images were analyzed using GenePix™ PRO 6.0 software. Local background subtracted median spot intensities were used for higher level data analysis using GeneSpring™ software from Agilent Technologies.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. Aspects of one embodiment may be applied to other embodiments and vice versa. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,617,060
U.S. Pat. No. 5,719,060
U.S. Pat. No. 6,225,047
U.S. Pat. No. 6,329,209
U.S. Pat. No. 6,344,334
U.S. Pat. No. 6,461,515
U.S. Pat. No. 6,475,391
U.S. Patent Application 20020137106
WO 00/56934
WO 98/59360
WO 98/59360
WO 99/51773
Baldini et al., J. Am. Chem. Soc. 126, 5656-5657, 2004.
Barany and Merrifield, In: The Peptides, Gross and Meienhofer (Eds.), Academic Press, NY, 1-284, 1979.
Barglow and Cravatt, Chem. Biol. 11, 1523-1531, 2004.
Beattie et al., Eur. J. Biochem. 239, 479-486, 1996.
Blackwell et al., Chem. Biol. 8, 1167-1182, 2001.
Brocchini et al., J Am. Chem. Soc., 119:4553-4554, 1997.
Cho et al., Bioorg Med Chem 7, 1171-1179, 1999.
Clemons et al., Chem. Biol. 8, 1183-1195, 2001.
Eichler et al., Med Res Rev., 15(6):481-96, 1995
Falsey et al., Bioconj. Chem. 12, 346-353, 2001.
Figliozzi et al., Methods Enzymol. 267, 437-447, 1996.
Fodor et al., Science 251, 767-773, 1991.
Frank, J. Immunol. Methods 267, 13-26, 2002.
Gallop et al., J Med. Chem., 37(9):1233-1251, 1994.
Goodey et al., J. Am. Chem. Soc. 123, 2559-2570, 2001.
Gordon et al., J. Med. Chem., 37(10):1385-401, 1994.
Harris et al., Chem. Biol. 8, 1131-1141, 2001.
Heine et al., Tetrahedron 59, 9919-9930, 2003.
Homak, In: The Basics of MRI, 2002.
Jellis et al., Gene 137, 63-68, 1993.
Kanemitsu, Comb Chem High Throughput Screen, 5(5):339-360, 2002.
Koehler et al., J. Am. Chem. Soc. 125, 8420-8421, 2003.
Kuruvilla et al., Nature 416, 653-657, 2002.
Lam and Renil, Curr. Opin. Chem. Biol. 6, 353-358, 2002.
LePlae et al., J Amer. Chem. Soc., 124:6820-6821, 2002.
Lesaicherre et al., Bioorg. Med. Chem. Lett. 12, 2085-2088, 2002.
Li et al., Chem. Commun., 581-583, 2005.
Li et al., J. Am. Chem. Soc. 126, 4088-4089, 2004.
MacBeath et al., J. Am. Chem. Soc. 121, 7967-7968, 1999.
Martin et al., Proteomics 3, 1244-1255, 2004.
Mason et al., Biochemistry 43, 6535-6544, 2004.
Merrifield, Science, 232(4748):341-347, 1986.
Olivos et al., Org. Lett. 4, 4057-4059, 2002.
Ostergaard and Holm, Mol. Divers., 3:17-27, 1997.
Petricoin, et al., J. Proteome Res. 3, 209-217, 2004.
Pweletz et al., Drug. Dev. Res. 49, 34-42, 2000.
Rakow and Suslick, Nature 406, 710-713, 2000.
Reddy and Kodadek, Proc. Nat. Acad. Sci. USA 102, 12672-12677, 2005.
Reddy et al., Chem. Biol. 11, 1127-1137, 2004.
Reineke et al., J. Immun. Methods 267, 37-51, 2002.
Shaginian et al., J. Am. Chem. Soc. 126, 16704-16705, 2004.
Simon et al., Proc. Natl. Acad. Sci. USA 89, 9367-9371, 1992.
Stewart and Young, In: Solid Phase Peptide Synthesis, 2d. ed., Pierce Chemical Co., 1984.
Takahashi et al., Chem. Biol. 10, 53-60, 2003.
Tam et al., J. Am. Chem. Soc., 105:6442, 1983.
Thompson and Ellman, Chem. Rev., 96(1):555-600, 1996.
Usui et al., Biopolyrners (Pept. Sci.) 76, 129-139, 2004.
Uttamchandani et al., Curr. Opin. Chem. Biol. 9, 4-13, 2005.
Winssinger et al., Proc. Natl. Acad. Sci. USA 99, 11139-11144, 2002.
Wulfkuhle et al., Nat. Rev. Cancer 3, 267-276, 2003.
Yang et al., J. Amer. Chem. Soc., 121:589-590, 1999.
Zuckermann et al., J. Am. Chem. Soc. 114, 10646-10647, 1992.

What is claimed is:

1. A method of profiling a plurality of distinct ligand binding moiety in a sample comprising:
(a) providing an array of ligands having a plurality of random structures;
(b) contacting said array with a biological sample comprising ligand binding moiety; and
(c) assessing binding of ligand binding moiety to said array, wherein binding of ligand binding moiety to said array detects ligand binding moieties in said sample.

2. The method of claim 1, wherein said ligand binding moiety is comprised in a body fluid or on a cell surface.

3. The method of claim 2, wherein the cell surface is on a non-adherent cell.

4. The method of claim 3, wherein the cell is a T cell.

5. The method of claim 1, wherein said ligand binding moiety is an antibody.

6. The method of claim 5, wherein assessing binding of the ligand binding moiety to the array features comprises contacting the array with labeled anti-Ig.

7. The method of claim 1, wherein said ligand binding moieties are enzymes.

8. The method of claim 7, wherein assessing comprises labeling the enzymes with fluorescently labeled or otherwise labeled mechanism-based inhibitors or other covalent inhibitors.

9. The method of claim 1, wherein said ligand binding moieties are nucleotide-binding proteins, glycosylated proteins, post-translationaly modified proteins, peptide hormones or ligands.

10. The method of claim 9, wherein assessing comprises detecting fluorescently or otherwise labeled nucleotides or nucleotide analogues, fluorescently or otherwise-labeled sugar-binding molecules, or fluorescently or otherwise-labeled antibodies.

11. The method of claim 1, wherein said random ligands comprise peptides, peptoids, oligonucleotides, oligosaccharides or small molecules.

12. The method of claim 1, wherein said random ligands are preselected based on known reactivity to said ligand binding moieties or a class of ligand binding moieties.

13. The method of claim 12, wherein the profile of binding of said ligand binding moiety is predictive of a disease state in a subject from which said sample was obtained.

14. The method of claim 13, wherein the disease state is selected from the group consisting of cancer, autoimmune disease, inflammatory disease, infectious disease, neurodegenerative disease, or cardiovascular disease.

15. The method of claim 14, wherein profile of binding differentiates between different forms of a disease state.

16. The method of claim 15, wherein the profile of binding differentiates forms of a disease state as mild or aggressive.

17. The method of claim 14, wherein the profile of binding differentiates between a disease state that is or is not responsive to a treatment or therapy.

18. The method of claim 14, wherein the disease state is breast cancer, lung cancer, prostate cancer, cervical cancer, head and neck cancer, testicular cancer, ovarian cancer, skin cancer, brain cancer, pancreatic cancer, liver cancer, stomach cancer, colon cancer, rectal cancer, esophageal cancer, lymphoma, or leukemia, such as antibodies that recognize epitopes unique to these disease states.

19. The method of claim 14, wherein autoimmune disease is lupus, myestenia gravis, multiple sclerosis, narcolepsy, rheumatoid arthritis, nephritis, Chagas disease, scleroderma, or Sjogren's disease.

20. The method of claim 14, wherein infection is a result of infection with viruses, bacteria or fungi.

21. The method of claim 14, wherein the neurodegenerative disease is Alzheimer's disease, dementia, or Creutzfeld-Jacob disease.

22. The method of claim 1, wherein said random ligands comprise a purely random feature and/or a non-random feature.

23. The method of claim 1, wherein said random ligands are not preselected based on known reactivity to said ligand binding proteins.

24. The method of claim 1, wherein said array comprises between about 1000 and 100,000 distinct random ligands.

25. The method of claim 1, wherein said array comprises between about 2000 and 50,000 distinct random ligands.

26. The method of claim 1, wherein said array comprises between about 4000 and 25,000 distinct random ligands.

27. The method of claim 1, wherein said array comprises between about 6000 and 15,000 distinct random ligands.

28. The method of claim 1, wherein said array comprises between about 7000 and 12,500 distinct random ligands.

29. The method of claim 1, wherein said array comprises between about 8000 and 10,000 distinct random ligands.

30. The method of claim 1, wherein said sample is urine, serum, whole blood, cerebrospinal fluid, sputum, saliva, or semen.

31. The method of claim 1, wherein said array is a microscope slide, plate, a chip, or a population of beads.

32. The method of claim 1, wherein said sample is from a cow, horse, chicken, or human subject.

33. The method of claim 1, further comprising cross-linking said ligand binding moiety to said array.

34. The method of claim 1, further comprising associating a ligand structure with binding to a ligand binding moiety.

35. The method of claim 1, further comprising assessing binding of a control ligand binding moiety to a ligand.

36. A method of profiling the binding of one or more isoforms of a ligand binding moiety in a sample comprising:
(a) providing an array of ligands having a plurality of random structures;
(b) contacting said array with a biological sample comprising one or more isoforms; and
(c) assessing binding of said one or more isoforms to said array,
wherein binding of said one or more isoforms detects said one or more isoforms in said sample.

37. The method of claim 36, wherein a plurality of isoforms are bound to the array, wherein specific isoforms are detected by specific binding patterns.

38. The method of claim 36, wherein said one or more isoforms are enzymes.

39. The method of claim 38, wherein assessing comprises photometric or non-photometric means.

40. The method of claim 36, wherein said one or more isoforms are phosphorylation isoforms, glycosylation isoforms, myristoylation isoforms, length isoforms, amino acid substitution isoforms, ubiquitylation isoforms, SUMOylation isoforms, NEDDylation isoforms, splice variants, methylation isoforms, acetylation isoforms, citrullation isoforms, nitrosylation isoforms, or formylation isoforms.

41. The method of claim 36, wherein said random ligands comprise peptides, peptoids, oligonucleotides, oligosaccharides, amino acid derivatives, or small molecules.

42. The method of claim 36, wherein said random ligands are preselected based on known reactivity to said isoforms.

43. The method of claim 42, wherein the pattern of binding of said one or more isoforms is predictive of a disease state in a subject from which said sample was obtained.

44. The method of claim 42, wherein the pattern of binding of said one or more isoforms is predictive of activation or inhibition of a cellular pathway.

45. The method of claim 36, wherein said random ligands are not preselected based on known reactivity to said one or more isoforms.

46. The method of claim 36, wherein said array comprises between about 1000 and 100,000 distinct random ligands.

47. The method of claim 36, wherein said array comprises between about 2000 and 50,000 distinct random ligands.

48. The method of claim 36, wherein said array comprises between about 4000 and 25,000 distinct random ligands.

49. The method of claim 36, wherein said array comprises between about 6000 and 15,000 distinct random ligands.

50. The method of claim 36, wherein said array comprises between about 7000 and 12,500 distinct random ligands.

51. The method of claim 36, wherein said array comprises between about 8000 and 10,000 distinct random ligands.

52. The method of claim 36, wherein said sample is urine, serum, whole blood, cerebrospinal fluid, sputum, saliva, or semen.

53. The method of claim 36, wherein said array is a plate, a chip, or a population of beads.

54. The method of claim 36, wherein said isoforms are phosphorylation isoforms, glycosylation isoforms, myristoylation isoforms, length isoforms, amino acid substitution isoforms, ubiquitylation isoforms, SUMOylation isoforms, NEDDylation isoforms, splice variants, methylation isoforms, acetylation isoforms, citrullation isoforms, nitrosylation isoforms, or formylation isoforms.

55. The method of claim 36, wherein said sample has been stimulated prior to step (a).

56. The method of claim 55, wherein said sample has been stimulated with a drug.

57. The method of claim 56, wherein said sample comprises cells stimulated in vitro.

58. The method of claim 56, wherein said sample is obtained from a subject suffering from a disease state.

59. The method of claim 55, wherein said sample has been stimulated with an environmental condition.

60. The method of claim 59, wherein said environmental condition is light, heat, cold, sleep deprivation, elevated noise, sound deprivation, light deprivation, or chemical exposure.

61. The method of claim 36, wherein said sample is from a human subject.

62. The method of claim 36, further comprising cross-linking said ligand binding proteins to said array.

63. The method of claim 36, further comprising associating a ligand structure with binding to an isoform.

64. The method of claim 36, further comprising assessing binding of more than one isoform to a single ligand.

65. The method of claim 36, wherein said random ligands comprise a purely random feature and a non-random feature.
